(19) United States
(12) Patent Application Publication
(10) Pub. No.: US 2008/0319001 A1
(43) Pub. Date: Dec. 25, 2008

(54) Cancer Treatment Using Specific 3,6,9-Substituted Acridines

(76) Inventors: Christina Martins, London (GB); Stephen Neidle, London (GB); Mekala Gunaratnam, London (GB); John Stuart, London (GB); Lloyd Kelland, London (GB)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/886,128

(22) PCT Filed: Mar. 2, 2006
(86) PCT No.: PCT/GB2006/000740

§ 371 Date: Mar. 28, 2008

(30) Foreign Application Priority Data
Mar. 11, 2005 (GB) 0505045.5

(51) Int. Cl. A61K 31/438; C07D 401/14; A61P 35/00

(52) U.S. Cl. 514/297; 546/105

(57) ABSTRACT

The present invention relates to a 3,6,9 acridine compound and optionally substituted derivatives thereof that may be useful in the treatment of cancer. The invention also provides compositions comprising the compounds and uses thereof. Formula (I) wherein each of R₁, R₂, R₃, R₄ and R₅ is either fluorine or is not present (i.e. represents a hydrogen atom); n represents 1 or 2.

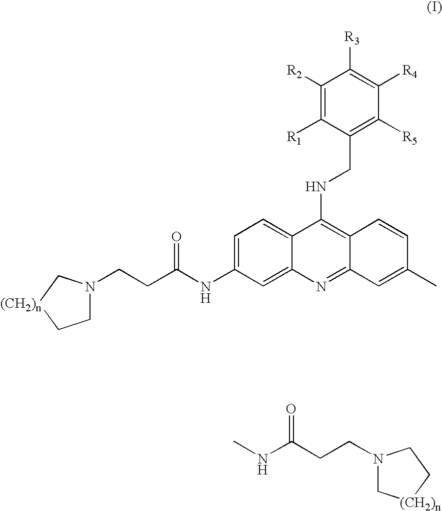

### FIELD OF THE INVENTION

This invention relates to the field of telomerase inhibitors and anti-proliferative agents, and more specifically to certain acridone and acridine compounds which inhibit telomerase and/or regulate cell proliferation. The present invention also relates to pharmaceutical compositions comprising such compounds, and the use of such compounds and compositions, both in vitro and in vivo, to inhibit telomerase and/or to regulate cell proliferation.

### BACKGROUND OF THE INVENTION

Mammalian cells are normally subject to tight controls regulating replication in order to maintain organ structure and function. However, there are a number of diseases including cancer that are characterised by uncontrolled cellular proliferation. Compromise of any of the steps involved in cell cycle regulation could be involved in a cell's escape from regulatory mechanisms and therefore lead to neoplasia. However, if a cell escapes the suppression of proliferation, there are limitations to the number of replicative cycles it can progress through before safety mechanisms cause the cell cycle to shutdown, and this restriction is thought to be a component of the process of organismal aging. Although aging is a complex process, a major candidate for the molecular signal for limiting cellular proliferation is that of telomere shortening.

Telomeres are nucleoprotein structures at the ends of linear chromosomes consisting of DNA sequences arranged in randomly repeated units which extend from less than 100 to several thousands of bases. In contrast to chromosome ends created by random breakage, telomeres are stable structures not prone to degradation or fusion with other chromosome ends and are not subject to normal DNA repair mechanisms.

During each round of cellular replication, both strands of double-stranded DNA separate and daughter strands are synthesised in a slightly different manner on the leading and lagging strand. While the lead strand replicates in a continuous fashion using conventional DNA polymerase, the lagging strand replicates in a discontinuous fashion using Okazaki fragments. The gaps between individual Okazaki fragments are filled in by the regular DNA polymerase. However, this sets the stage for a potential “end replication problem.” This arises because Okazaki fragment priming will not necessarily start at the very end of the DNA and because the RNA primer, once removed, would result in a portion of unreplicated 3′-DNA (an unrepaired 3′-overhang). This can lead to a loss of 50-200 base pairs with every round of somatic cell division, with eventual shortening of telomeres to a length that coincides with the activation of an anti-proliferative mechanism termed “mortality stage 1” (M1), and at this stage, senescence in somatic cells occurs. Thus, telomere shortening functions as a “mitotic clock” and limits division in somatic cells to about 50-70 times, thereby contributing to cell aging.

In some cells, due to various mechanisms, the M1 stage is bypassed and cells can continue to divide until telomeres become critically shortened (“mortality stage 2” or “M2”). During the M2 stage, a specialised DNA polymerase called “telomerase” is found in many immortalised cells. Telomerase utilises its internal RNA template to synthesize the telomeric sequence and compensate for the loss of telomeric DNA due to incomplete replication. This prevents further shortening of telomeres, and the resulting stabilization of their length contributes to immortalization.

Telomerase is not normally expressed in healthy mammalian somatic cells, or if it is, its activity is repressed. Telomerase is however expressed in certain regenerative cell lines including male germ line cells and some epithelial stem cells (e.g., as in the intestinal crypts, the basal layer of the epidermis, and within human hair follicles).

Active telomerase molecules and shortened but stabilised telomeres have been detected in the majority of tumours examined (and in over 90% of all human cancers examined), and consequently, telomeres and telomerase are recognised targets for anti-neoplastic (e.g., cancer) chemotherapy.

The absence of telomerase in most healthy cells makes this enzyme a particularly attractive target, considering that its inhibition would probably cause minimal damage to the patient as a whole. The fact that tumour cells have shorter telomeres and higher proliferation rates than normal replicative cell populations suggests that a therapeutic telomerase inhibitor may contribute to tumour cell death well before damage to regenerative tissues occurs, thereby minimizing undesirable side-effects.

Telomerase also has a role in protecting (‘capping’) the ends of telomeres in cancer cells. Disruption of this function may lead to destabilisation of telomere maintenance in tumour cells, with the consequence of a rapid onset of senescence, followed by apoptosis of these cells. It has been suggested that G-quadruplex ligands are able to selectively uncap telomeres in tumour cells (Leonetti et al., 2004), as shown by the ability of the tri-substituted acridine compound BR-ACO-19 to cause end-to-end fusions in chromosomes from a tumour cell line (Incles et al, 2004). Evidence to date suggests that targeting of telomeres in tumour cells and consequent disruption of telomere maintenance is a particular property of G-quadruplex ligands, and is more important than telomerase inhibition per se, in order for these compounds to have antitumour activity in vitro and in vivo.

For a more detailed discussion of telomeres and telomerase, and their role as anti-proliferative targets, see, for example, Sharma et al., 1997; Urquidi et al., 1998; Perry et al., 1998c; Autexier, 1999; Neidle et al., 1999; Neidle and Parkinson, 2002; Shay and Wright, 2002; Mergny et al., 2002, and references therein.

A number of polycyclic compounds, including polycyclic acridines, anthraquinones, and fluorenones have been shown to inhibit telomerase and/or to have anti-tumour effects in vitro. See, for example, Bostock-Smith et al., 1999;

Gimenez-Arnau et al., 1998; Gimenez-Arnau et al., 1998; Hagan et al., 1997; Hagan et al., 1998; Harrison et al., 1999; Julino et al., 1998; Perry et al., 1998a, 1998b, 1999a, 1999b; Sun et al., 1997.

Harrison et al., 1999, describe certain 3,6-disubstituted acridines which are shown to inhibit telomerase, and to inhibit cell growth in certain ovarian carcinoma cell lines.

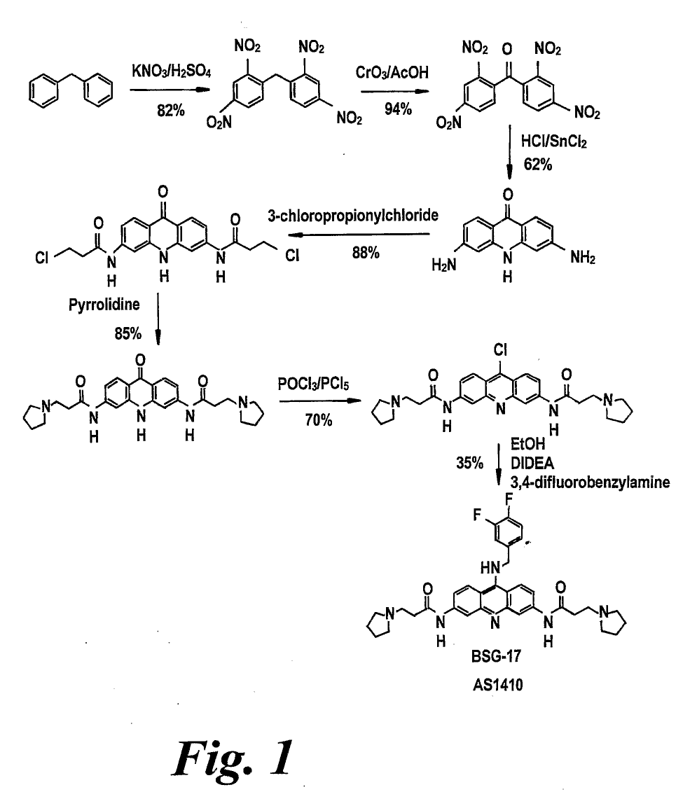

Read et al., April 2001; Harrison et al., 2003; and Schultes et al., 2004, describe certain 3,6,9-trisubstituted acridines (see compounds 3 and 4 in FIG. 1 therein), including BR-ACO-19 (BSG01) and BR-ACO-20 which are shown to have potent in vitro inhibitory activity against human telomerase. Burger et al, 2005, describe the in vivo activity of BR-ACO-19 (BSG01) in a human tumour xenograft model.

Although a number of telomerase inhibitors are known, there remains a need for effective telomerase inhibitors and anti-tumour agents, and in particular for telomerase inhibitors which offer additional pharmacological advantages.

For example, particularly preferred telomerase inhibitors are ones which are characterised by one or more of the following properties.
(a) Wide differential between in vitro telomerase inhibition (nM) vs. inhibition of Taq polymerase (>10 micromolar) (in order to provide specificity and eliminate broad-spectrum polymerase inhibitors); preferably no inhibition of Taq polymerase at <5 μM.
(b) Wide differential (>10-fold) between cell-free telomerase inhibition (nM vs acute cytotoxicity (micromolar); preferably cell free telomerase inhibition at <0.5 μM.
(c) Shortening of telomere length in tumour cells at concentrations 5 to 10-fold less than concentrations for acute cytotoxicity.
(d) Induction of senescence or apoptosis in human tumour cells at concentrations at least 5-fold less than acute cytotoxicity.
(e) Acceptable in vivo plasma half life (desired >2 hours).
(f) In vivo anti-tumour activity in mice at non-toxic doses in 2-3 independent tumour models by intravenous route.
(g) Acceptable “pharmaceutical” properties (solubility >1 mg/ml in biocompatible ideally aqueous-based solvent; stability >6 hours), synthesis amenable to scaling-up.
(h) Telomere shortening in human tumour xenografts.
(i) Potential oral bioavailability.
(j) Ability to inhibit the growth of tumour cells at concentrations significantly less than concentrations for acute cytotoxicity.
(k) Ability to bind to human quadruplex DNA with high affinity such that the increase in melting temperature is >10° C.
(l) Possession of lipophilic structural features that may aid cellular uptake.

### DESCRIPTION OF THE INVENTION

The present invention therefore relates to a specific member and optionally substituted derivatives thereof, of a class of compounds referred to herein, as “acridines”.

In a first aspect of the invention there is provided a 3,6,9 acridine compound of formula I:

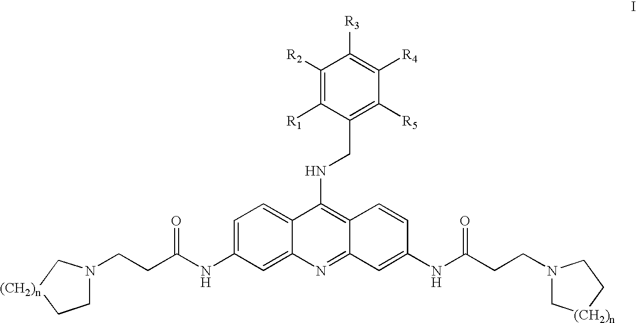

wherein each of R₁, R₂, R₃, R₄ and R₅ is either fluorine or is not present (i.e. represents a hydrogen atom);
n represents, on each occasion when used herein, 1 or 2; and
which compound is optionally substituted at one or more positions by substituents independently selected from:
halo; hydroxy; ether (e.g., C₁₋₇alkoxy); imino; oxo; formyl; acyl (e.g., C₁₋₇alkylacyl, C₅₋₂₀arylacyl); acylhalide; carboxy; ester; acyloxy; amido; acylamido; thioamido; tetrazolyl; amino; nitro; nitroso; azido; cyano; isocyano; cyanato; isocyanato; thiocyano; isothiocyano; sulfhydryl; thioether (e.g., C₁₋₇alkylthio); sulphonic acid; sulphonate; sulphone; sulphonyloxy; sulphonyloxy; sulphamino; sulphonamino; sulphinamino; sulphamyl; sulphonamido; C₁₋₇alkyl (including, e.g., C₁₋₇haloalkyl, C₁₋₇hydroxyalkyl, C₁₋₇carboxyalkyl, C₁₋₇aminoalkyl, C₅₋₂₀aryl-C₁₋₇alkyl); C₃₋₂₀heterocyclyl; and C₅₋₂₀aryl (including, e.g., C₅₋₂₀carboaryl, C₅₋₂₀heteroaryl, C₁₋₇alkyl-C₅₋₂₀aryl and C₅₋₂₀haloaryl),
and pharmaceutically acceptable derivatives thereof,
which compounds are hereinafter referred to as “compounds of the invention”.

By “pharmaceutically acceptable derivatives” we include pharmaceutically acceptable salts, esters, amides, solvates, hydrates and protected forms thereof.

The compounds of the invention may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl.

However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Specifically included in the term “isomer” are compounds with one or more isotopic substitutions. For example, H may be in any isotopic form, including ¹H, ²H (D), and ³H (T); carbon may be in any isotopic form, including ¹²C, ¹³C, and ¹⁴C; oxygen may be in any isotopic form, including ¹⁶ O and ¹⁸O.

Unless otherwise specified, a reference to the compounds of the invention includes all such isomeric forms, including (wholly or partially) racemic and other mixtures thereof. Methods for the preparation (e.g., asymmetric synthesis) and separation (e.g., fractional crystallisation and chromatographic means) of such isomeric forms are known in the art and/or include the methods taught in the examples herein.

The compounds of the invention can exist in a number of different resonance structures, of which only one is illustrated above. A reference to one resonance structure is a reference to all possible corresponding resonance structures.

It may be convenient or desirable to prepare, purify, and/or handle corresponding salts of the active compounds, for example, pharmaceutically acceptable salts.

Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH⁺) and substituted ammonium ions (e.g., NH₃R⁺, NH₂R₂⁺, NHR₃⁺, NR₄⁺).

Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous.

Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicylic, sulphanilic, 2-acetyoxybenzoic, fumaric, toluenesulphonic, methanesulphonic, ethane disulphonic, oxalic, isethionic, and valeric.

Examples of suitable polymeric anions include, but are not limited to, those derived from the following polymeric acids: tannic acid, carboxymethyl cellulose.

It may be convenient or desirable to prepare, purify, and/or handle a corresponding solvate of the active compounds. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compounds in a chemically protected form. By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T Green and P Wuts, Wiley, 1991)

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases, as an N-oxide (>NO^{˜}).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇allyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

For the avoidance of doubt, in cases in which the identity of two or more substituents in a compound of the invention may be the same, the actual identities of the respective substituents are not in any way interdependent. For example, in the situation in which the optional substituents are both alkyl groups, then the alkyl groups may be the same or different.

Compounds of the invention that may be mentioned include those in which:

both n values are the same;
n represents 1; and/or
the optional substituents are independently selected from halo; hydroxy; ether (e.g., C₁₋₇alkoxy); formyl; acyl (e.g., C₁₋₇alkylacyl, C₅₋₂₀arylacyl); acylhalide; carboxy; ester; acyloxy; amido; acylamido; thioamido; tetrazolyl; amino; nitro; nitroso; azido; cyano; isocyano; cyanato; isocyanato; thiocyano; isothiocyano; sulphydryl; thioether (e.g., C₁₋₇alkylthio); sulphonic acid; sulphonate; sulphone; sulphonyloxy; sulphinyloxy; sulphamino; sulphonamino; sulphonamino; sulphamyl; sulphonamido; C₁₋₇alkyl (including, e.g., C₁₋₇haloalkyl, C₁₋₇hydroxyalkyl, C₁₋₇carboxyalkyl, C₁₋₇aminoalkyl, C₅₋₂₀aryl-C₁₋₇alkyl); C₃₋₂₀heterocyclyl; and C₅₋₂₀aryl (including, e.g., C₅₋₂₀carboaryl, C₅₋₂₀heteroaryl, C₁₋₇allyl-C₅₋₂₀aryl and C₅₋₂₀haloaryl).

Then the compounds of formula I are substituted by the optional substituents (i.e. the substituents other than R₁ to R₅) the embodiments of the invention that may be mentioned are as follows.

In a first embodiment, the optional substituents are, when present, attached at any available position in the compounds of formula I. That is, the substituents may be attached to:
(1) the nitrogen heterocycle at the terminal ends of the 3- and 9-substituents of the acridine (i.e. the pyrrolidine or piperidine heterocycles of the compounds of the invention);
(2) the linker group between the nitrogen heterocycles mentioned at (1) above and the 3- and 9-position of the acridine (i.e. the N(H)—C(O)—(CH₂)₂— linker groups);
(3) the acridine ring system (i.e. at the 2-, 4-, 5-, 7-, 8- or 10-position of the acridine);
(4) the —CH₂— group of the benzyl group at the 6-position of the acridine ring; and/or
(5) the positions of phenyl ring of the compounds of formula I at which no fluoro group is present (i.e. when any one or more of R₁, R₂, R₃, R₄ and R₅, represents other than fluoro).

In a second embodiment, the optional substituents are, when present, attached at the positions detailed at (1) to (4) above (i.e. to any position on the compounds of formula I other than to the C atoms to which the groups R₁ to R₅ are attached).

It is preferred that the optional substituents, when present, are attached at the positions outlined at (1), (3) and, in the case of compounds of the first embodiment of the invention, (5) above.

In an alternative (and particularly preferred) embodiment of the invention, the optional substituents are absent (and thus R₁ to R₅ are the only substituents of the compounds of formula I).

Methods for formation and introduction of the optional substituents into a variety of parent groups are also well known.

Each of the optional substituents listed above are now described in more detail:

C₁₋₇ alkyl

The term “C₁₋₇alkyl,” as used herein, pertains to a monovalent moiety obtained by removing a hydrogen atom from a C₁₋₇hydrocarbon compound having from 1 to 7 carbon atoms, which may be aliphatic or alicyclic, or a combination thereof, and which may be saturated, partially unsaturated, or fully unsaturated (but not aromatic).

Examples of (unsubstituted) saturated linear C₁₋₇alkyl groups include, but are not limited to, methyl, ethyl, n-propyl, n-butyl, and n-pentyl (amyl).

Examples of (unsubstituted) saturated branched C₁₋₇alkyl groups include, but are not limited to, iso-propyl, iso-butyl, sec-butyl, tert-butyl, and neo-pentyl.

Examples of saturated alicyclic (carbocyclic) C₁₋₇allyl groups (also referred to as “C₃₋₇cycloalkyl” groups) include, but are not limited to, unsubstituted groups such as cyclopropyl, cyclobutyl, cyclopentyl, and cyclohexyl, as well as substituted groups (e.g., groups which comprise such groups), such as part cyclic groups or cyclic groups substituted by alkyl groups e.g. methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

Examples of (unsubstituted) unsaturated C₁₋₇alkyl groups which have one or more carbon-carbon double bonds (also referred to as “C₂₋₇alkenyl” groups) include, but are not limited to, ethenyl (vinyl, —CH═CH₂), 2-propenyl (allyl, —CH—CH═CH₂), isopropenyl (—C(CH₃)═CH₂), butenyl, pentenyl, and hexenyl.

Examples of (unsubstituted) unsaturated C₁₋₇alkyl groups which have one or more carbon-carbon triple bonds (also referred to as “C₁₋₇alkynyl” groups) include, but are not limited to, ethynyl (ethinyl) and 2-propynyl (propargyl).

Examples of unsaturated alicyclic (carbocyclic) C₁₋₇alkyl groups which have one or more carbon-carbon double bonds (also referred to as “C₃₋₇cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl and cyclohexenylmethyl.

C₃₋₂₀heterocyclyl

The term “C₃₋₂₀heterocyclyl” as used herein, pertains to a monovalent moiety obtained by removing a hydrogen atom from a ring atom of a C₃₋₂₀heterocyclic compound, said compound having one ring, or two or more rings (e.g., spiro, fused, bridged), and having from 3 to 20 ring atoms, atoms, of which from 1 to 10 are ring heteroatoms, and wherein at least one of said ring(s) is a heterocyclic ring. Preferably, each ring has from 3 to 7 ring atoms, of which from 1 to 4 are ring heteroatoms. “C₃₋₂₀” denotes ring atoms, whether carbon atoms or heteroatoms.

Examples of (non-aromatic) monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);
O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);
S₁: thiirane (C₃), thietane (C₄), thiolane (tetrahydrothiophene) (C₅), thiane (tetrahydrothiopyran) (C₆), thiepane (C₇);
O₂: dioxolane (C₅), dioxane (C₆), and dioxepane (C₇);
O₃: trioxane (C₆);
N₂; imidazolidine (C₅), pyrazolidine (diazolidine) (C₅), imidazoline (C₅), pyrazoline (dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane (thioxane) (C₆); and,
N₁O₁S₁: oxathiazine (C₆)

Examples of substituted (non-aromatic) monocyclic heterocyclyl groups include saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Examples of heterocyclyl groups which are also heteroaryl groups are described below with aryl groups

C₅₋₂₀aryl

The term “C₅₋₂₀aryl,” as used herein, pertains to a monovalent moiety obtained by removing a hydrogen atom from an aromatic ring atom of a C₅₋₂₀aromatic compound, said compound having one ring, or two or more rings (e.g., fused), and having from 5 to 20 ring atoms, and wherein at least one of said ring(s) is an aromatic ring. Preferably, each ring has from 5 to 7 (e.g. 5 or 6) ring atoms.

In this context, the prefixes (e.g., C₃₋₂₀, C₅₋₇, C₅₋₆, etc.) denote the number of ring atoms, or range of number of ring atoms, whether carbon atoms or heteroatoms. For example, the term “C₅₋₆aryl,” as used herein, pertains to an aryl group having 5 or 6 ring atoms. Examples of groups of aryl groups include C₃₋₂₀aryl, C₅₋₇aryl, and C₅₋₆aryl.

The ring atoms may be all carbon atoms, as in “carboaryl groups” (e.g., C₅₋₂₀carboaryl).

Examples of carboaryl groups include, but are not limited to, those derived from benzene (i.e., phenyl) (C₆), naphthalene (C₁₀), azulene (C₁₀), anthracene (C₁₄), phenanthrene (C₁₄), naphthacene (C₁₈), and pyrene (C₁₆). Such groups include fused rings in which all the rings are aromatic.

Examples of aryl groups which comprise fused rings, at least one of which is an aromatic ring, include, but are not limited to, groups derived from indene (C₉), isoindene (C₉), and fluorene (C₁₃).

Alternatively, the ring atoms may include one or more heteroatoms, including but not limited to oxygen, nitrogen, and sulphur, as in “heteroaryl groups.” In this case, the group may conveniently be referred to as a “C₅₋₂₀heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of monocyclic heteroaryl groups include, but are not limited to, those derived from:

N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₃S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅)

Examples of heterocyclic groups (some of which are also heteroaryl groups) which comprise fused rings, include, but are not limited to:

C₉heterocyclic groups (with 2 fused rings) derived from benzofuran (O₁), isobenzofuran (O₁), indole (N₁), isoindole (N₁), purine (N4) (e.g., adenine, guanine), benzimidazole (N₂), benzoxazole (N₂O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀heterocyclic groups (with 2 fused rings) derived from benzodioxan (O₂), quinoline (N₁), isoquinoline (N₁), benzoxazine (N₁O₁), benzodiazine (N₂), pyridopyridine (N₂), quinoxaline (N₂), quinazoline (N₂);
C₁₃heterocyclic groups (with 3 fused rings) derived from carbazole (N₁), dibenzofuran (O₁), dibenzothiophene (S₁); and,
C₁₄heterocyclic groups (with 3 fused rings) derived from acridine (N₁), xanthene (O₁), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂)
Heterocyclic groups (including heteroaryl groups) which have a nitrogen ring atom in the form of an —NH— group may be N-substituted, that is, as —NR^{a}—. For example, pyrrole may be N-methyl substituted, to give N-methylpyrrole. Examples of such N-substitutents (i.e. values of R^{a}) include, but are not limited to C₁₋₇alkyl, C₃₋₂₀heterocyclyl, C₅₋₂₀aryl, and acyl groups
Heterocyclic groups (including heteroaryl groups) which have a nitrogen ring atom in the form of an —N— group may be substituted in the form of an N-oxide, that is, as —N(═O)— (also denoted —N(->O)—). For example, quinoline may be substituted to give quinoline N-oxide; pyridine to give pyridine N-oxide; benzofurazan to give benzofurazan N-oxide (also known as benzofuroxan).

Cyclic groups may additionally bear one or more oxo (═O) groups on ring carbon atoms. Monocyclic examples of such groups include, but are not limited to, those derived from:

C₅: cyclopentanone, cyclopentenone, cyclopentadienone;
C₆: cyclohexanone, cyclohexenone, cyclohexadienone;
O₁: furanone (C₅), pyrone (C₆);
N₁: pyrrolidone (pyrrolidinone) (C₅), piperidinone (piperidone) (C₆), piperidinedione (C₆);
N₂: imidazolidone (imidazolidinone) (C₅), pyrazolone (pyrazolinone) (C₅), piperazinone (O₆), piperazinedione (C₆), pyridazinone (C₆), pyrimidinone (C₆) (e.g., cytosine), pyrimidinedione (C₆) (e.g., thymine, uracil), barbituric acid (C₆);
N₂S₁: thiazolone (C₅), isothiazolone (C₅);
N₁O₁: oxazolinone (C₅);

Polycyclic examples of such groups include, but are not limited to, those derived from:

C₉: indanedione;
N₁: oxindole (C₉);
O₁: benzopyrone (e.g., coumarin, isocoumarin, chromone) (C₁₀);
NO₁O₁: benzoxazolinone (C₉), benzoxazolinone (C₁₀);
N₂: quinazolinedione (C₁₀);
N₄: purinone (C₉) (e.g., guanine)

Still more examples of cyclic groups which bear one or more oxo (═O) groups on ring carbon atoms include, but are not limited to, those derived from:

cyclic anhydrides (—C(═O)—O—C(═O)— in a ring), including but not limited to maleic anhydride (C₅), succinic anhydride (C₅), and glutaric anhydride (C₆);
cyclic carbonates (—O—C(═O)—O— in a ring), such as ethylene carbonate (C₅) and 1,2-propylene carbonate (C₅);
imides (—C(═O)—NR—C(═O)— in a ring), including but not limited to, succinimide (C₅), maleimide (C₅), phthalimide, and glutarimide (C₆);
lactones (cyclic esters, —O—C(═O)— in a ring), including, but not limited to, β-propiolactone, γ-butyrolactone, δ-valerolactone (2-piperidone), and ε-caprolactone;
lactams (cyclic amides, —NR^{b}—C(═O)— in a ring), including, but not limited to, β-propiolactam (C₄), γ-butyrolactam (2-pyrrolidone) (C₅), δ-valerolactam (C₆), and ε-caprolactam (C₇);
cyclic carbamates (—O—C(═O)—NR^{c}— in a ring), such as 2-oxazolidone (C₅);
cyclic ureas (—NR^{d}—C(═O)—NR— in a ring), such as 2-imidazolidone (C₅) and pyrimidine-2,4-dione (e.g., thymine, uracil) (C₆);
in which the N-substituents (i.e. R^{b}, R^{c}, R^{d} and R^{e}) preferably, and independently, represent C₁₋₇alkyl, C₃₋₂₀heterocyclyl, C₅₋₂₀aryl groups and acyl groups.

The above C₁₋₇alkyl, C₃₋₂₀heterocyclyl, and C₅₋₂₀aryl groups, whether alone or part of another substituent, may themselves optionally be substituted with one or more groups selected from themselves and the additional substituents listed below.

### Hydrogen: —H

Note that if the substituent at a particular position is hydrogen, it may be convenient to refer to the compound as being “unsubstituted” at that position.

### Halo

—F, —Cl, —Br, and —I

### Hydroxy

—OH

### Ether

An ether substituent is one in which an oxygen atom is between two carbon atoms. Accordingly, it may be a —R^{f}—O—R^{g} group or, more preferably, an —OR^{h} group, wherein R^{f}, R^{g} and R^{h} independently represent an ether substituent, for example, a C₁₋₇alkyl group (also referred to as a C₁₋₇alkoxy group, discussed below), a C₃₋₂₀heterocyclyl group (also referred to as a C₃₋₂₀heterocyclyloxy group), or a C₅₋₂₀aryl group (also referred to as a C₅₋₂₀aryloxy group), and preferably a C₁₋₇alkyl group.

C₁₋₇alkoxy

—ORⁱ, wherein Rⁱ is a C₁₋₇alkyl group. Examples of C₁₋₇alkoxy groups include, but are not limited to, —OCH₃ (methoxy), —OCH₂CH₃ (ethoxy) and —OC(CH₃)₃ (tert-butoxy).

Oxo (keto, -one): —O

Examples of cyclic compounds and/or groups having, as a substituent, an oxo group (═O) include, but are not limited to, carbocyclics such as cyclopentanone and cyclohexanone; heterocyclics, such as pyrone, pyrrolidone, pyrazolone, pyrazolinone, piperidone, piperidinedione, piperazinedione, and imidazolidone; cyclic anhydrides, including but not limited to maleic anhydride and succinic anhydride; cyclic carbonates, such as propylene carbonate; imides, including but not limited to, succinimide and maleimide; lactones (cyclic esters, —O—C(═O)— in a ring), including, but not limited to, β-propiolactone, γ-butyrolactone, δ-valerolactone, and ε-caprolactone; and lactams (cyclic amides, —NH—C(═O)— in a ring), including, but not limited to, β-propiolactam, γ-butyrolactam, δ-valerolactam, and ε-caprolactam.

### Imino (Imine)=

═NR^{j}, wherein R^{j} is an imino substituent, for example, hydrogen, C₁₋₇alkyl group, a C₃₋₂₀heterocyclyl group, or a C₅₋₂₀aryl group, preferably hydrogen or a C₁₋₇alkyl group. Examples of ester groups include, but are not limited to, ═NH, ═NMe, ═NEt, and ═NPh.

### Formyl (Carbaldehyde, Carboxaldehyde)

—C(═O)H

### Acyl (Keto)

—C(═O)R^{k}, wherein R^{k} is an acyl substituent, for example, a C₁₋₇alkyl group (also referred to as C₁₋₇alkylacyl or C₁₋₇alkanoyl), a C₃₋₂₀heterocyclyl group (also referred to as C₃₋₂₀heterocyclylacyl), or a C₅₋₂₀aryl group (also referred to as C₅₋₂₀arylacyl), preferably a C₁₋₇alkyl group. Examples of acyl groups include, but are not limited to, —C(═O)CH₃ (acetyl), —C(═O)CH₂CH₃ (propionyl), —C(═O)C(CH₃)₃ (pivaloyl), and —C(═O)Ph (benzoyl, phenone).

### Acylhalide (Haloformyl, Halocarbonyl)

—C(═O)X, wherein X is —F, —Cl, —Br, or —I, and preferably —Cl, —Br, or —I

### Carboxy (Carboxylic Acid)

—COOH

### Ester (Carboxylate, Carboxylic Acid Ester, Oxycarbonyl)

—C(═O)OR^{m} wherein R^{m} is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.

### Acyloxy (Reverse Ester)

—OC(═O)Rⁿ, wherein Rⁿ is an acyloxy substituent, for example, a C₁₋₇alkyl group, a C₃₋₂₀heterocyclyl group, or a C₅₋₂₀aryl group, preferably a C₁₋₇alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

### Amido (Carbamoyl, Carbamyl, Aminocarbonyl, Carboxamide)

—C(═O)NR^{p}R^{q}, wherein R^{P} and R^{q} are independently amino substituents, for example, hydrogen, a C₁₋₇alkyl group, a C₃₋₂₀heterocyclyl group, or a C₅₋₂₀aryl group, and, preferably, H or a C₁₋₇alkyl group, or, R^{P} and R^{q} may be linked together to form a “cyclic” amino group, i.e. R^{P} and R^{q}, taken together with the nitrogen atom to which they are attached, form a heterocyclic ring having from 3 (e.g. 4) to 8 (e.g. 6) ring atoms (such as from 4 to 8 ring atoms), which heterocyclic ring, for example, optionally contains a further (in addition to the essential nitrogen atom to which R^{P} and R^{q} are necessarily attached) one or two (e.g. one) heteroatoms (e.g. nitrogen, oxygen or sulphur). Examples of amido groups include, but are not limited to, —C(═O)NH₂, —C(═O)NHCH₃, —C(═O)NH(CH₃)₂, —C(═O)NHCH₂CH₃, and —C(═O)N(CH₂CH₃)₂, as well as amido groups in which R^{P} and R^{q}, together with the nitrogen atom to which they are attached, form a heterocyclic structure, for example, piperidinocarbonyl, morpholinocarbonyl, thiomorpholinocarbonyl, and piperazinocarbonyl.

### Acylamido (Acylamino)

—NR^{r}C(═O)R^{s}, wherein R^{r} is an amide substituent, for example, a C₁₋₇alkyl group, a C₃₋₂₀heterocyclyl group, or a C₅₋₂₀aryl group, and preferably a C₁₋₇alkyl group, and R^{s} is an acyl substituent, for example, a C₁₋₇alkyl group, a C₃₋₂₀heterocyclyl group, or a C₅₋₂₀aryl group, and preferably a C₁₋₇alkyl group. Examples of acylamide groups include, but are not limited to, —NHC(═O)CH₃, —NHC(═O)CH₂CH₃, and —NHC(═O)Ph. R^{r} and R^{s} may together form a cyclic structure, as in, for example, succinimidyl, maleimidyl, and phthalimidyl.

### Thioamido (Thiocarbamyl)

—C(═S)NR^{t}R^{u}, wherein R^{t} and R^{u} are independently amino substituents, for example, hydrogen, a C₁₋₇alkyl group, a C₃₋₂₀heterocyclyl group, or a C₅₋₂₀aryl group, and, preferably, H or a C₁₋₇alkyl group, or, R^{t} and R^{u} may be linked together to form a “cyclic” amino group, i.e. R^{t} and R^{u}, taken together with the nitrogen atom to which they are attached, form a heterocyclic ring having from 3 (e.g. 4) to 8 (e.g. 6) ring atoms (such as from 4 to 8 ring atoms), which heterocyclic ring, for example, optionally contains a further (in addition to the essential nitrogen atom to which R^{t} and R^{u} are necessarily attached) one or two (e.g. one) heteroatoms (e.g. nitrogen, oxygen or sulphur). Examples of amido groups include, but are not limited to, —C(═S)NH₂, —C(═S)NHCH₃, —C(═S)NH(CH₃)₂, and —C(═S)NHCH₂CH₃.

### Tetrazolyl

A five membered aromatic ring having four nitrogen atoms and one carbon atom.

### Amino

—NR^{v}R^{w}, wherein R^{v} and R^{w} are independently amino substituents, for example, hydrogen, a C₁₋₇alkyl group (also referred to as C₁₋₇alkylamino or di-C₁₋₇alkylamino), a C₃₋₂₀heterocyclyl group, or a C₅₋₂₀aryl group, preferably H or a C₁₋₇alkyl group, or, R^{v} and R^{w} may be linked together to form a “cyclic” amino group, i.e. R^{v} and R^{w}, taken together with the nitrogen atom to which they are attached, form a heterocyclic ring having from 3 (e.g. 4) to 8 (e.g. 6) ring atoms (such as from 4 to 8 ring atoms), which heterocyclic ring, for example, optionally contains a further (in addition to the essential nitrogen atom to which R^{P} and R^{q} are necessarily attached) one or two (e.g. one) heteroatoms (e.g. nitrogen, oxygen or sulphur). Examples of amino groups include, but are not limited to, —NH₂, —NHCH₃, —NHCH(CH₃)₂, —N(CH₃)₂, —N(CH₂CH₃)₂, and NHPh. Examples of cyclic amino groups include, but are not limited to, aziridino, azetidino, piperidino, piperazino, morpholino, and thiomorpholino.

### Nitro

—NO₂

### Nitroso

—NO

### Azido

—N₃

Cyano (nitrile carbonitrile)

### —CN

### Isocyano

—NC

### Cyanato

—OCN

### Isocyanato

—NCO

### Thiocyano (Thiocyanato)

—SCN

Isothiocyano (isothiocyanato)

### —NCS

### Sulphydryl (Thiol, Mercapto)

—SH

### Thioether (Sulphide)

A thioether substituent is one in which a sulfur atom is between two carbon atoms. Accordingly, it may be a —R^{x}—S—R^{y} group or, more preferably, an —SR^{z} group, wherein R^{x}, R^{y} and R^{z} independently represent a thioether substituent, for example, a C₁₋₇alkyl group (also referred to as a C₁₋₇alkylthio group), a C₃₋₂₀heterocyclyl group, or a C₅₋₂₀aryl group, and preferably a C₁₋₇allyl group. Examples of C₁₋₇alkylthio groups include, but are not limited to, —SCH₃ and —SCH₂CH₃.

### Sulphonic Acid (Sulpho)

—S(═O)₂OH

### Sulphonate (Sulphonic Acid Ester)

S(═O)₂OR^{aa}, wherein R^{aa} is a sulphonate substituent, for example, a C₁₋₇alkyl group, a C₃₋₂₀heterocyclyl group, or a C₅₋₂₀aryl group, and preferably a C₁₋₇alkyl group. Examples of sulphonate groups include, but are not limited to, —S(═O)₂OCH₃ and —S(═O)₂OCH₂CH₃.

### Sulphone (Sulphonyl)

—S(═O)₂R^{ab}, wherein R^{ab} is a sulphone substituent, for example, a C₁₋₇alkyl group, a C₃₋₂₀heterocyclyl group, or a C₅₋₂₀aryl group, and preferably a C₁₋₇alkyl group. Examples of sulphone groups include, but are not limited to, —S(═O)₂CH₃ (methanesulphonyl, mesyl), —S(═O)₂CF₃, —S(═O)₂CH₂CH₃, and 4-methylphenylsulphonyl (tosyl).

### Sulphonyloxy

—OS(═O)₂R^{ac}, wherein R^{ac} is a sulphonyloxy substituent, for example, a C₁₋₇alkyl group, a C₃₋₂₀heterocyclyl group, or a C₁₋₂₀aryl group, and preferably a C₁₋₇alkyl group. Examples of sulphonyloxy groups include, but are not limited to, —OS(═O)₂CH₃ and —OS(═O)₂CH₂CH₃.

### Sulphinyloxy

—OS(═O)R^{ad}, wherein R^{ad} is a sulphinyloxy substituent, for example, a C₁₋₇alkyl group, a C₃₋₂₀heterocyclyl group, or a C₅₋₂₀aryl group, and preferably a C₁₋₇alkyl group. Examples of sulphinyloxy groups include, but are not limited to, —OS(═O)CH₃ and —OS(═O)CH₂CH₃.

### Sulphamino

—NR^{ae}S(═O)₂OH, wherein R^{ae} is an amino substituent, for example, hydrogen, a C₁₋₇alkyl group, a C₃₋₂₀heterocyclyl group, or a C₅₋₂₀aryl group, and preferably H or a C₁₋₇alkyl group. Examples of sulphamino groups include, but are not limited to, —NHS(═O)₂OH and —N(CH₃)S(═O)₂OH.

### Sulphonamino

—NR^{af}S(═O)₂R^{ag}, wherein R^{af} is an amino substituent, for example, hydrogen, a C₁₋₇alkyl group, a C₃₋₂₀heterocyclyl group, or a C₅₋₂₀aryl group, and preferably H or a C₁₋₇alkyl group, and R^{ag} is a sulphonamino substituent, for example, a C₁₋₇alkyl group, a C₃₋₂₀heterocyclyl group, or a C₅₋₂₀aryl group, and preferably a C₁₋₇alkyl group. Examples of sulphonamino groups include, but are not limited to, —NHS(═O)₂CH₃ and —N(CH₃)S(═O)₂C₆H₅.

Sulphinamino

—NR^{ah}S(═O)R^{ai}, wherein R^{ah} is an amino substituent, for example, hydrogen, a C₁₋₇alkyl group, a C₃₋₂₀heterocyclyl group, or a C₅₋₂₀aryl group, and preferably H or a C₁₋₇alkyl group, and R^{ai} is a sulphinamino substituent, for example, a C₁₋₇alkyl group, a C₃₋₂₀heterocyclyl group, or a C₅₋₂₀aryl group, and preferably a C₁₋₇alkyl group. Examples of sulphinamino groups include, but are not limited to, —NHS(═O)CH₃ and —N(CH₃)S(═O)C₆H₅.

### Sulphamyl

—S(═O)NR^{aj}R^{ak}, wherein R^{aj} and R^{ak} are independently amino substituents, for example, hydrogen, a C₁₋₇alkyl group (also referred to as C₁₋₇alkylamino or di-C₁₋₇alkylamino), a C₃₋₂₀heterocyclyl group, or a C₅₋₂₀aryl group, preferably H or a C₁₋₇alkyl group, or, R^{aj} and R^{ak} may be linked together to form a “cyclic” amino group, i.e. R^{aj} and R^{ak}, taken together with the nitrogen atom to which they are attached, form a heterocyclic ring having from 3 (e.g. 4) to 8 (e.g. 6) ring atoms (such as from 4 to 8 ring atoms), which heterocyclic ring, for example, optionally contains a further (in addition to the essential nitrogen atom to which R^{aj} and R^{ak} are necessarily attached) one or two (e.g. one) heteroatoms (e.g. nitrogen, oxygen or sulphur) Examples of sulphamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

### Sulphonamido

—S(═O)₂NR^{am}R^{an}, wherein R^{am} and R^{an} are independently amino substituents, as defined for amino groups. Examples of sulphonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

### Further Substitutions

As mentioned above, a C₁₋₇alkyl group may be substituted with, for example, hydroxy (also referred to as a C₁₋₇hydroxyalkyl group), C₁₋₇alkoxy (also referred to as a C₁₋₇alkoxyalkyl group), amino (also referred to as a C₁₋₇aminoalkyl group), halo (also referred to as a C₁₋₇haloalkyl group), carboxy (also referred to as a C₁₋₇carboxyalkyl group), and C₅₋₂₀aryl (also referred to as a C₅₋₂₀aryl-C₁₋₇alkyl group).

Similarly, a C₅₋₂₀aryl group may be substituted with, for example, hydroxy (also referred to as a C₅₋₂₀hydroxyaryl group), halo (also referred to as a C₅₋₂₀haloaryl group), amino (also referred to as a C₅₋₂₀-aminoaryl group, e.g., as in aniline), C₁₋₇allyl (also referred to as a C₁₋₇alkyl-C₅₋₂₀aryl group, e.g., as in toluene), and C₁₋₇alkoxy (also referred to as a C₁₋₇alkoxy-C₅₋₂₀aryl group, e.g., as in anisole).

These and other specific examples of such substituted groups are also discussed below:

C₁₋₇haloalkyl Group

The term “C₁₋₇haloalkyl group,” as used herein, pertains to a C₁₋₇alkyl group in which at least one hydrogen atom (e.g., 1, 2, 3) has been replaced with a halogen atom (e.g., F, Cl, Br, I). If more than one hydrogen atom has been replaced with a halogen atom, the halogen atoms may independently be the same or different. Every hydrogen atom may be replaced with a halogen atom, in which case the group may conveniently be referred to as a C₁₋₇ perhaloalkyl group.

Examples of C₁₋₇haloalkyl groups include, but are not limited to, —CF₃, —CHF₂, —CH₂F, —CCl₃, —CBr₃, —CH₂CH₂F, —CH₂CHF₂, and —CH₂CF₃.

C₁₋₇hydroxyalkyl

The term “C₁₋₇hydroxyalkyl group,” as used herein, pertains to a C₁₋₇allyl group in which at least one hydrogen atom has been replaced with a hydroxy group.

Examples of C₁₋₇hydroxyalkyl groups include, but are not limited to, —CH₂OH, —CH₂CH₂OH, and —CH(OH)CH₂OH.

C₁₋₇carboxyalkyl

The term “C₁₋₇carboxyalkyl group,” as used herein, pertains to a C₁₋₇alkyl group in which at least one hydrogen atom has been replaced with a carboxy group. Examples of C₁₋₇carboxyalkyl groups include, but are not limited to, —CH₂COOH and —CH₂CH₂COOH.

C₁₋₇aminoalkyl

The term “C₁₋₇aminoalkyl group,” as used herein, pertains to a C₁₋₇alkyl group in which at least one hydrogen atom has been replaced with an amino group. Examples of C₁₋₇aminoalkyl groups include, but are not limited to, —CH₂NH₂, —CH₂CH₂NH₂, and —CH₂CH₂N(CH₃)₂.

C₁₋₇alkyl-C₅₋₂₀aryl

The term “C₁₋₇alkyl-C₅₋₂₀aryl,” as used herein, describes certain C₅₋₂₀aryl groups which have been substituted with a C₁₋₇alkyl group. Examples of such groups include, but are not limited to, tolyl (as in toluene), xylyl (as in xylene), mesityl (as in mesitylene), styryl (as in styrene), and cumenyl (as in cumene).

C₅₋₂₀aryl-C₁₋₇alkyl

The term “C₅₋₂₀aryl-C₁₋₇alkyl,” as used herein, describers certain C₁₋₇alkyl groups which have been substituted with a C₅₋₂₀aryl group. Examples of such groups include, but are not limited to, benzyl (phenylmethyl), tolylmethyl, phenylethyl, and triphenylmethyl (trityl).

C₅₋₂₀haloaryl

The term “C₅₋₂₀haloaryl,” as used herein, describes certain C₅₋₂₀aryl groups which have been substituted with one or more halo groups. Examples of such groups include, but are not limited to, halophenyl (e.g., fluorophenyl, chlorophenyl, bromophenyl, or iodophenyl, whether ortho-, meta-, or para-substituted), dihalophenyl, trihalophenyl, tetrahalophenyl, and pentahalophenyl.

Embodiments of the invention that may be mentioned include those in which, in the compounds of formula I in which the optional substituents are present:
(A) R^{a}, R^{b}, R^{c}, R^{d} and R^{e} independently represent C₅₋₆ heterocyclyl, C₅₋₆ aryl (e.g. phenyl) or —C(O)—C₁₋₃ alkyl or, more preferably C₁₋₆ (e.g. C₁₋₄) alkyl (such as ethyl or, more particularly, methyl);
   when any one of R^{f}, R^{g}, R^{h}, Rⁱ, R^{k}, R^{m}, R^{r}, R^{s}, R^{x}, R^{y}, R^{z}, R^{aa}, R^{ab}, R^{ac}, R^{ad}, R^{ag} and R^{ai} represents C₁₋₇ alkyl then that alkyl group may be substituted by a hydroxy, amino, carboxy, or, more particularly, C₅₋₆ aryl (e.g. phenyl) or halo, or is more preferably, unsubstituted.
   R^{f}, R^{g}, R^{h}, Rⁱ, R^{k}, R^{m}, R^{r}, R^{s}, R^{x}, R^{y}, R^{z}, R^{aa}, R^{ab}, R^{ac}, R^{ad}, R^{ag} and R^{ai} independently represent a C₅₋₈ (e.g. C₅ or C₆) heterocyclyl group, a C₅₋₈ (e.g. C₅ or C₆) aryl group or, preferably, a C₁₋₆ alkyl group;
   R^{p}, R^{q}, R^{t}, R^{u}, R^{v}, R^{w}, R^{ae}, R^{af}, R^{ah}, R^{aj}, R^{ak}, R^{am}, R^{an}, independently represent a C₅₋₈ (e.g. C₅ or C₆) heterocyclyl group, a C₅₋₈ (e.g. C₅ or C₆) aryl group or, preferably, a C₁₋₆ (e.g. C₁₋₃) alkyl group or the relevant pairs (i.e. R^{P} and R^{q}, R^{t} and R^{u}, R^{v} and R^{w}, R^{aj} and R^{ak}, and R^{am} and R^{an}) are linked as defined herein;
(B) R^{h} represents C₁₋₄ alkyl (e.g. methyl, ethyl or tert-butyl), which group is optionally substituted by one or two (e.g. one) C₅₋₆ aryl groups (e.g. phenyl) or halo (e.g. fluoro, chloro or bromo);
   R^{j1} represents hydrogen, C₁₋₃ alkyl (e.g. methyl or ethyl) or C₅₋₆ aryl (e.g. phenyl);
   R^{k} and R^{m} independently represent C₁₋₄ alkyl (e.g. methyl, ethyl or tert-butyl) or C₅₋₆ aryl (e.g. phenyl);
   Rⁿ represents C₁₋₄ alkyl (e.g. methyl, ethyl or tert-butyl), which group is optionally substituted by one or two (e.g. one) C₅₋₆ aryl group (e.g. phenyl), or C₅₋₆ aryl (e.g. phenyl);
   R^{P} and R^{q} independently represent hydrogen, C₁₋₃ alkyl (e.g. methyl or ethyl) or are linked together as hereinbefore defined;
   R^{r} represents hydrogen;
   R^{s} represents C₁₋₃ alkyl (e.g. methyl or ethyl) or C₅₋₆ aryl (e.g. phenyl), or
   R^{r} and R^{s} are linked as hereinbefore defined;
   R^{V} and R^{w} independently represent hydrogen, C₁₋₄ allyl (e.g. methyl, ethyl, n-propyl, isopropyl, n-butyl or t-butyl) or C₅₋₆ aryl (e.g. phenyl), or are linked together as hereinbefore defined;
   when the optional substituent(s) is/are (an) unsubstituted alkyl group(s),
   then that/those group(s) represent(s) C₁₋₄ alkyl (e.g. methyl, ethyl, n-propyl, isopropyl, n-butyl or t-butyl);
   when the optional substituent(s) is/are (a) substituted alkyl group(s), then those further substituents are selected from halo (e.g. fluoro, chloro or bromo), hydroxy and amino (e.g. —NH₂ or —NH(CH₃));
   when the optional substituent(s) is/are (a) C₁₋₇haloalkyl group(s), then that/those group(s) represent(s) C₁₋₂ alkyl substituted by one, two or three chloro, fluoro or bromo atoms;
   when the optional substituent(s) is/are (a) C₁₋₇hydroxyalkyl group(s), then that/those group(s) represent(s) C₁₋₂ alkyl substituted by one or two hydroxy groups;
   when the optional substituent(s) is/are (a) C₁₋₇aminoalkyl group(s), then that/those group(s) represent(s) C₁₋₂ alkyl substituted by one or two (e.g. one) amino groups as hereinbefore defined;
   when the optional substituent(s) is/are (a) C₅₋₂₀aryl group(s), then that/those group(s) represent(s) phenyl; and
(C) the optional substituent(s), are independently selected from halo, —OH, —OR^{h}, —SH, —SR^{z}, —C(O)H, —C(O)R^{k}, —C(O)OR^{m}, —C(O)NR^{p}R^{q}, NR^{r}C(O)R^{s}, —NR^{v}R^{w}, —CN, —NO₂, C₁₋₆ alkyl optionally substituted by one or more substituents selected from halo, hydroxy and amino as defined herein, and C₅₋₆ aryl.

In a particular embodiment of the invention, the optional substituent(s), may be any independently selected from the group containing:

### —F, —Cl, —Br, and —I;

### —OH;

### —OMe, —OEt, —O(tBu), and —OCH₂Ph;

### —SH;

### —SMe, —SEt, —S(tBu), and —SCH₂Ph;

### —C(═O)H;

### —C(═O)Me, —C(═O)Et, —C(═O)(tBu), and —C(═O)Ph;

### —C(═O)OH;

### —C(═O)OMe, —C(═O)OEt, and —C(═O)O(tBu);

### —C(═O)NH₂, —C(═O)NHMe, —C(═O)NMe₂, and —C(═O)NHEt;

—NHC(═O)Me, —NHC(═O)Et, —NHC(═O)Ph, succinimidyl, and maleimidyl;

### NH₂, —NHMe, —NHEt, —NH(iPr), —NH(nPr), —NMe₂, —NEt₂, —N(iPr)₂, —N(nPr)₂, —N(nBu)₂, and —N(tBu)₂;

### —CN;

### —NO₂;

-Me, -Et, -nPr, -iPr, -nBu, -tBu;
—CF₃, —CHF₂, —CH₂F, —CCl₃, —CBr₃, —CH₂CH₂F, —CH₂CHF₂, and —CH₂CF₃;
—OCF₃, —OCHF₂, —OCH₂F, —OCCl₃, —OCBr₃, —OCH₂CH₂F, —OCH₂CHF₂, and —OCH₂CF₃;

### —CH₂OH, —CH₂CH₂OH, and —CH(OH)CH₂OH;

—CH₂NH₂, —CH₂CH₂NH₂, and —CH₂CH₂NMe₂; and,
optionally substituted phenyl.

In a more particular embodiment of the invention, compounds of formula I that may be mentioned include those in which:

the optional substituent(s) are selected from halo, —OR^{h}, C₁₋₂ alkyl (e.g. methyl) optionally substituted by one or more fluoro atoms (so forming, for example, a trifluoromethyl group) and —NR^{v}R^{w};
the optional substituent(s) is/are on the nitrogen heterocycle (as outlined at point (1) above) or, in the case of the first embodiment of the invention, on the phenyl ring of the benzyl group (as outlined at point (5) above).

In a yet more particular embodiment of the invention, compounds of formula I that may be mentioned include those in which the optional substituent(s) are any independently selected from —F, —OMe, —CF₃, —NH₂ and —N(Me)₂.

Preferred compounds of the invention include those in which:

n represents 2 (thereby forming a piperidine group, optionally substituted at the 4-position by methyl) or, particularly, 1;
the acridine tricycle has no optional substituents;
the —N(H)—(CH₂)₂ linker groups have no optional substituents;
the —CH₂— linker group has no optional substituents;
at least one (e.g. one to three and, preferably, two) of R₁ to R₅ (e.g. R₃, preferably, R₃ and R₅ or, more preferably, R₃ and R₄) represent(s) F and the others represent hydrogen.

Most preferably the compound of formula I has the structure:

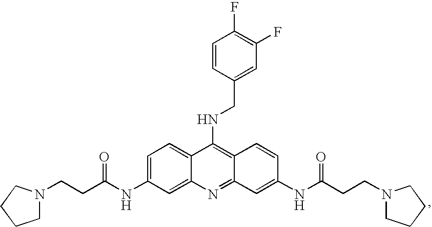

i.e. [N-(9-(3,4-Difluoro-benzylamino)-6-(3-pyrolidin-1-yl-propionamide)-acridin-3-yl)-3-pyrrolidin-1-yl-propionamide], alternatively referred to as BSG17.

Other specific embodiments of the invention that may be mentioned include:

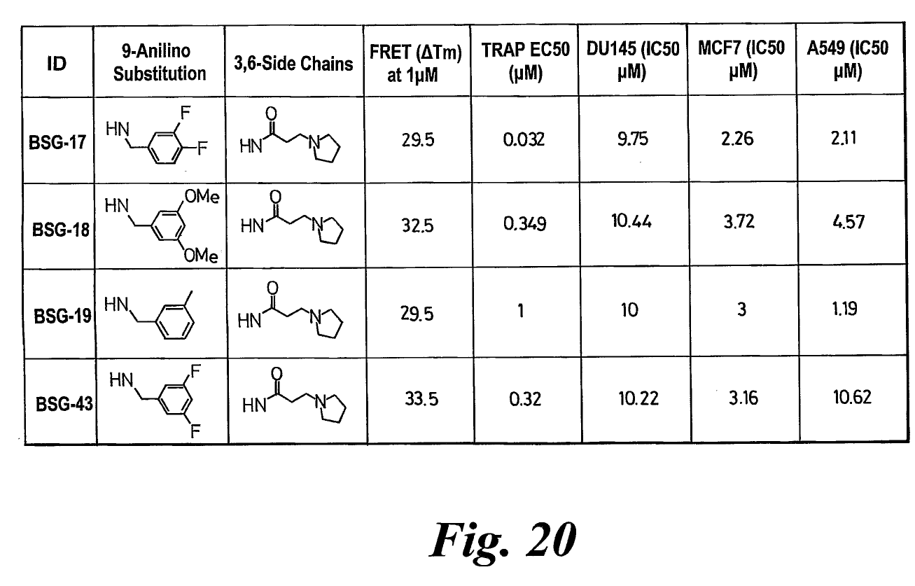

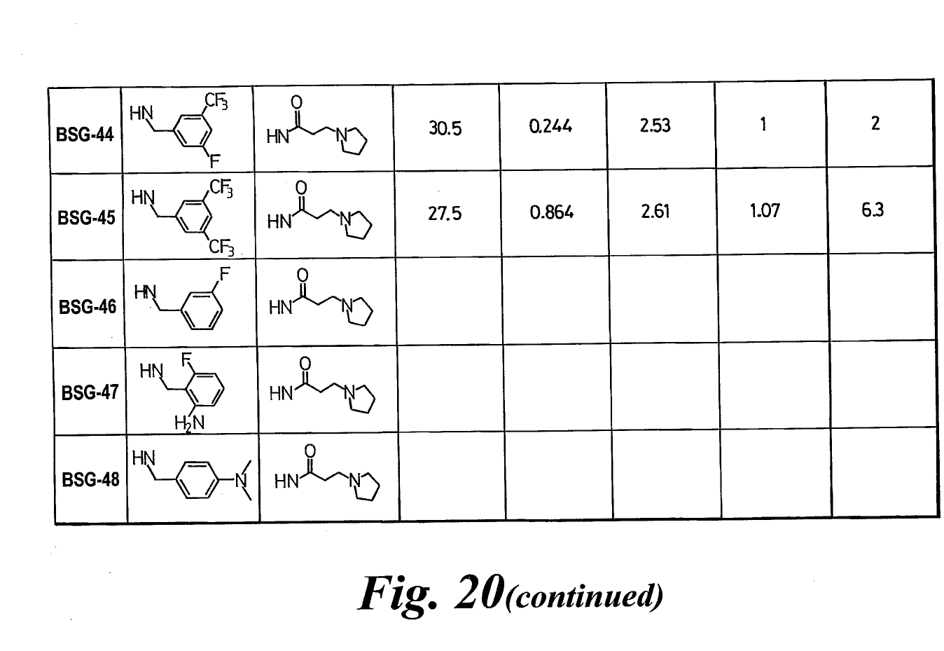

(I) the compounds of FIG. 20 in which no optional substituents are present (i.e. BSG-43, BSG-46 and BSG-22), which compounds have the following structures:

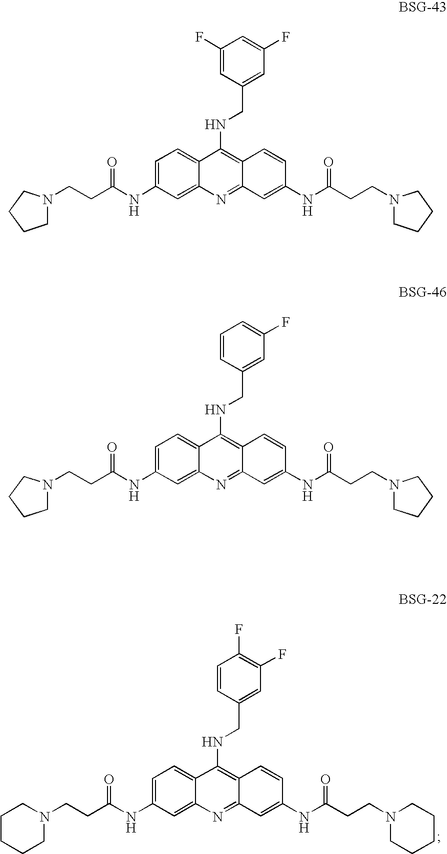

and
(II) the compounds of FIG. 20 in which optional substituents are present (i.e. BSG-18, BSG-19, BSG-44, BSG-45, BSG-47, BSG-48, BSG-20, BSG-21 and BSG-24), which compounds have the following structures:

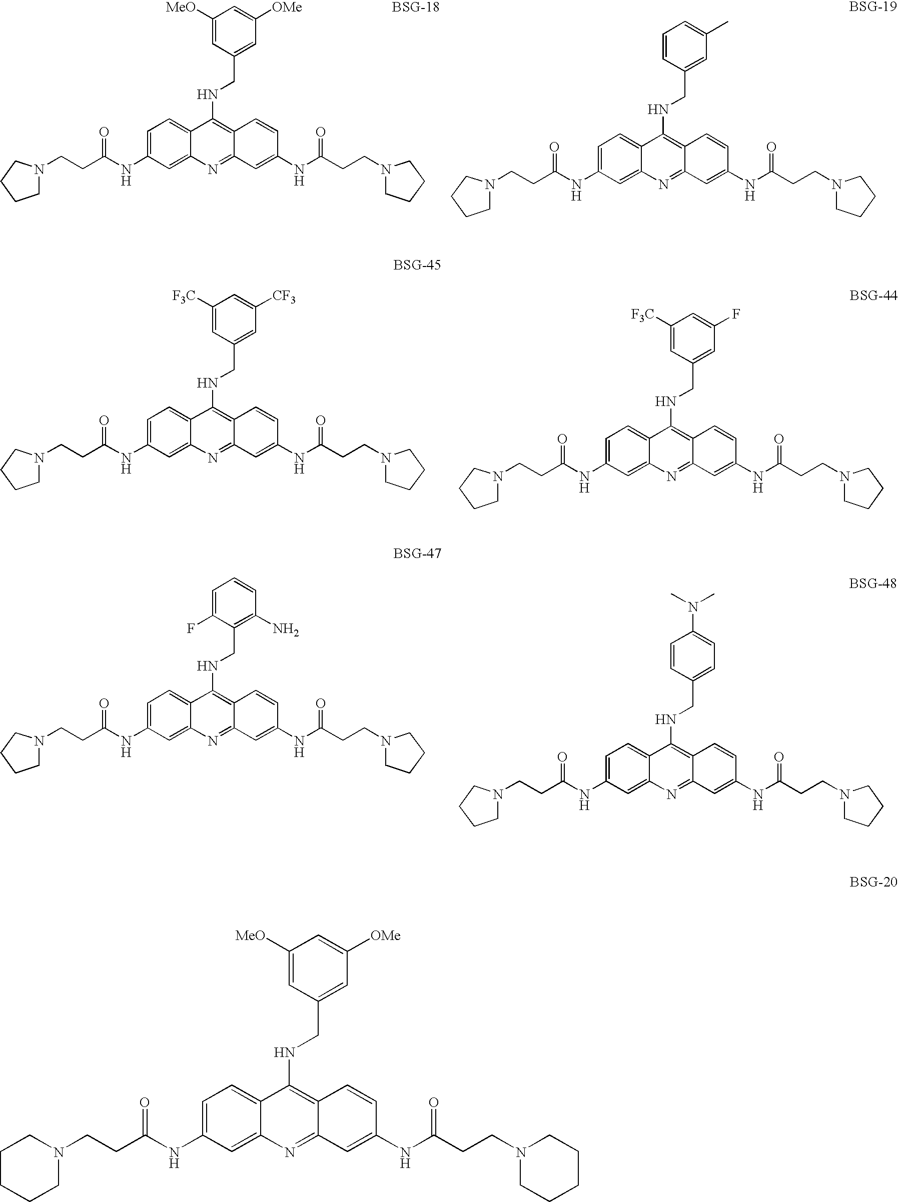

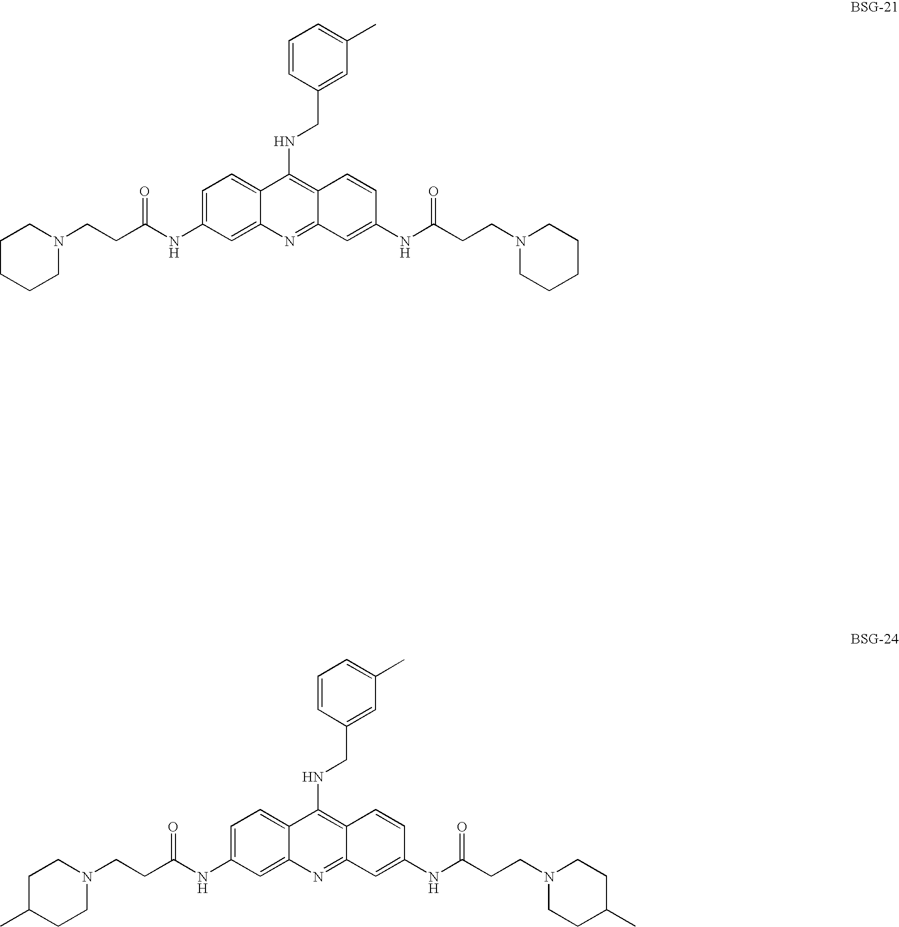

### Other Compound Forms

Included in the above are the well-known ionic, salt, solvate, and protected forms of these substituents. For example, a reference to carboxylic acid (—COOH) also includes the anionic (carboxylate) form (—COO⁻), a salt or solvate thereof, as well as conventional protected forms.

Similarly, a reference to an amino group includes the protonated form (—N⁺HRR), a salt or solvate of the amino group, for example, a hydrochloride salt, as well as conventional protected forms of an amino group.

Similarly, a reference to a hydroxy group also includes the anionic form (═O), a salt or solvate thereof, as well as conventional protected forms of a hydroxy group.

It may be convenient or desirable to prepare, purify, and/or handle the active compound in the form of a prodrug. Typically, the prodrug is inactive, or less active than the active compound, but may provide advantageous handling, administration, or metabolic properties.

For example, some prodrugs are esters of the active compound (e.g., a physiologically acceptable metabolically labile ester). During metabolism, the ester group (—C(═O)OR^{m}) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection, if required. Examples of such metabolically labile esters include those wherein R^{m} is C₁₋₇alkyl (e.g., -Me, -Et); C₁₋₇aminoalkyl (e.g., aminoethyl; 2-(N,N-diethylamino)ethyl; 2-(4-morpholino)ethyl); and acyloxy-C₁₋₇alkyl (e.g., acyloxymethyl; acyloxyethyl; e.g., pivaloyloxymethyl; acetoxymethyl; 1-acetoxyethyl; 1-(1-methoxy-1-methyl)ethyl-carbonxyloxyethyl; 1-(benzoyloxy)-ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxy-ethyl).

Thus, the compounds of the invention may conveniently be provided in a prodrug form.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

In a second aspect of the invention, there is provided a composition comprising the compound(s) described by the first aspect of the invention and a pharmaceutically acceptable excipient, diluent or carrier.

In a third aspect of the invention there are provided uses of the compound(s) of the first aspect or the composition of the second aspect.

Conveniently, the compound(s) and/or composition(s) may be used in the inhibition of telomerase. Active compounds may be used as cell culture additives to inhibit telomerase, for example, in order to regulate cell proliferation.

Therefore, the compound(s) and/or composition(s) may also be used in the regulation of cell proliferation.

Preferably, the compound(s) and/or composition(s) may be used as a medicament. Advantageously, the medicament may be used in the treatment and/or prevention and/or diagnosis of a disease characterised by increased cell-proliferation.

One of ordinary skill in the art is readily able to determine whether or not a candidate compound inhibits telomerase activity, regulates cell proliferation or exhibits anti-proliferative properties in relation to any particular cell line. For example, assays which may conveniently be used to assess the activity offered by a particular compound are described in the examples.

For example, a sample of cells (e.g., from a tumour) may be grown in vitro and an active compound brought into contact with said cells, and the effect of the compound on those cells observed. As examples of “effect,” the morphological status of the cells may be determined, or the expression levels of genes associated with cell cycle regulation determined. Where the active compound is found to exert an influence on the cells, this may be used as a prognostic or diagnostic marker of the efficacy of the compound in methods of treating a patient carrying cells of the same cellular type.

Examples of proliferative conditions include, but are not limited to, benign, pre-malignant, and malignant cellular proliferation, including but not limited to, neoplasms and tumours (e.g., histocytoma, glioma, astrocytoma, osteoma), cancers (e.g., ovarian carcinoma, breast carcinoma, bowel cancer, colon cancer, renal cancer, lung cancer, small cell lung cancer, testicular cancer, prostate cancer, sarcoma, osteosarcoma, Kaposi's sarcoma, melanoma), leukaemias, psoriasis, bone diseases, fibroproliferative disorders (e.g., of connective tissues), and atherosclerosis. Any type of cell may be treated, including but not limited to, colon, kidney (renal), breast (mammary), lung, ovarian, liver (hepatic), pancreas, skin, and brain.

The disease characterised by increased cell proliferation may be cancer.

Preferably the cancer is histocytoma, glioma, astrocytoma, osteoma, ovarian carcinoma, breast carcinoma, bowel cancer, colon cancer, renal cancer, lung cancer, small cell lung cancer, non-small cell lung cancer, testicular cancer, prostate cancer, sarcoma, osteosarcoma, Kaposi's sarcoma, melanoma, leukaemias, lymphomas, pancreatic cancer, skin cancer, cervical cancer and cancer of the esophagus.

The anti-cancer effect of the compounds of the invention may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

The invention further provides active compounds for use in a method of treatment of the human or animal body, for example, in the treatment of a proliferative condition, for example cancer. Such a method may comprise administering to such a subject a therapeutically-effective amount of an active compound, preferably in the form of a pharmaceutical composition.

Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; and gene therapy.

Preferably the medicament comprises a therapeutically or prophylactically effective dose, or plurality of doses, of the compound and/or composition.

Conveniently, the ideally effective dose is between 1 and 500 mg/m² in patients. Alternatively, the ideally effective dose may be expressed as between about 0.008 to about 4 mg/kg/day.

In a fourth aspect of the invention, there is provided the use of the compound(s) of the first aspect or the composition of the second aspect in the manufacture of a medicament for the treatment and/or prevention and/or diagnosis of a disease characterised by increased cell-proliferation.

In a fifth aspect of the invention there is provided the use of the compound(s) of the first aspect or the composition of the second aspect in an in vitro assay.

Active compounds may also be used as part of an in vitro assay, for example, in order to determine whether a candidate host is likely to benefit from treatment with the compound in question.

Active compounds may also be used as a standard, for example, in an assay, in order to identify other active compounds, other telomerase inhibitors, other anti-proliferative agents, etc.

Conveniently, the compound(s) and/or composition(s) may be used in an in vitro assay as a comparative standard in the identification of compounds that inhibit telomerase and/or regulate cell proliferation.

Alternatively, the compound(s) and/or composition(s) may be used in an in vitro assay to identify suitable candidate patients for later therapy.

In a sixth aspect of the invention there is provided a method of inhibiting telomerase and/or regulating cell proliferation including the step of contacting a cell with an effective amount of a compound of the first aspect or the composition of the second aspect.

Preferably the effective amount is between 0.1 to 10 micromolar (e.g. a 0.1 to 10 μM solution of the compound of formula I).

In a seventh aspect of the invention there is provided a method of synthesising a compound according to the first aspect of the invention.

Preferably the method comprises the steps shown in FIG. 1.

For example, compounds of formula I may be synthesised by reaction of a compound of formula II,

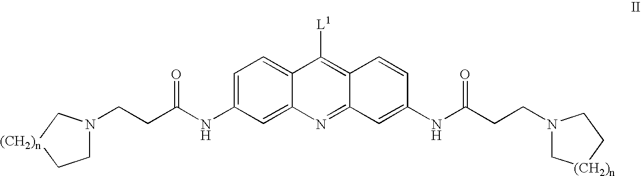

wherein L¹ represents a suitable leaving group such as halo (e.g. chloro) and n is as hereinbefore defined, with a compound of formula III,

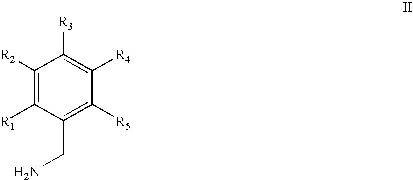

wherein R₁ to R₅ are as hereinbefore defined, in the presence of a suitable base (e.g. an amine base such as diisopropylethylamine) and an appropriate solvent (e.g. an alcoholic solvent (such as ethanol)).

Compounds of formula II and III are either commercially available, are known in the literature, or may be obtained either by analogy with the processes described herein (for example in accordance with FIG. 1), or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

In an eighth aspect of the invention there is provided a kit comprising
(a) the compound of the first aspect or the composition of the second aspect; and
(b) instructions for use.

Preferably the compound and/or composition is provided in a suitable container and/or with suitable packaging and the instructions are written instructions on how to administer the active compound.

The written instructions may also include a list of indications for which the active ingredient is a suitable treatment.

Preferably the kit also comprises means of administering the compound and/or composition. Conveniently such means of administering is a syringe or intravenous drip.

As stated above, the compounds of the invention may be administered alone, or, more preferably by way of known pharmaceutical formulations such as those described herein. As such, the compounds of the invention may be administered as the sole therapeutic agent.

However, according to a ninth aspect of the invention, compounds of formula I may also be combined with other therapeutic agents that are useful in the treatment of a disease in which inhibition of telomerase activity or regulation of cell proliferation (e.g. a proliferative condition) is desired and/or required. The same applies to related compounds having the same mode of action (e.g. compounds disclosed in WO 02/08193), which may be represented as compounds of formula Ia:

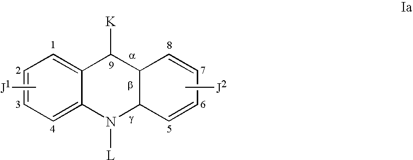

wherein either:
(a) K is ═O, L is —H, α is a single bond, β is a double bond, γ is a single bond (i.e., “acridones”); or:
(b) K is a 9-substituent, L is absent, α is a double bond, β is a single bond, γ is a double bond (i.e., “acridines”); and
J¹ and J² independently represent —N(H)—C(O)—(CH₂)₂—NR^{1a}R^{2a}:
n represents an integer from 1 to 5;
K represents —N(R^{N})Q;
R^{1a}, R^{2a} and R^{N} independently represent H, C₁₋₇alkyl, C₃₋₂₀heterocyclyl or C₅₋₂₀aryl, which latter three groups may be optionally substituted by one or more substituents selected from unsubstituted C₁₋₇alkyl, C₃₋₂₀heterocyclyl, C₅₋₂₀aryl and the list of optional substituents hereinbefore defined in respect of compounds of formula I; and
Q represents C₁₋₇alkyl, C₃₋₂₀heterocyclyl or C₅₋₂₀aryl, all of which may be optionally substituted as defined above,
   and pharmaceutically acceptable derivatives thereof.

Preferred compounds of formula Ia include those in which:

J¹ is a 2- or 3-substituent;
J² is a 6- or 7-substituent;
n represents from 1 to 4 (e.g. from 1 to 3 (such as 1 or 2));
R^{1a} and R^{2a} independently represent H, C₁₋₄ alkyl or are linked together to form a C₅₋₈heterocyclyl group (i.e. a 5 to 8-membered cyclic nitrogen heterocycle);
R^{N} represents H or C₁₋₇ (e.g. C₁₋₃) alkyl.

Particular compounds of formula Ia that may be mentioned include BSG-01 (also referred to as BRACO-19), which has the following structure:

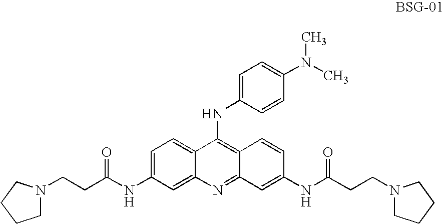

Accordingly, there is further provided a combination product comprising:
(A) a compound of formula I or Ia as hereinbefore defined, or a pharmaceutically acceptable derivative thereof, and
(B) a platin, or a pharmaceutically acceptable derivative thereof,
   wherein each of components (A) and (B) is formulated in admixture with a pharmaceutically-acceptable adjuvant, diluent or carrier.

The term “platin” as used herein, includes references to carboplatin or, preferably, cisplatin. For the avoidance of doubt, ciplatin is cis-diaminedichloroplatinum(II) (and is also represented below), and carboplatin is an analogue of cisplatin and is represented below.

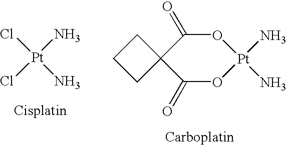

Such combination products provide for the administration of a compound of formula I or Ia in conjunction with the platin, and may thus be presented either as separate formulations, wherein at least one of those formulations comprises a compound of formula I or Ia, and at least one comprises the platin, or may be presented (i.e. formulated) as a combined preparation (i.e. presented as a single formulation including a compound of formula I or Ia and the platin).

Thus, there is further provided:
(1) a pharmaceutical formulation including a compound of formula I or Ia, as hereinbefore defined, a platin, and a pharmaceutically-acceptable adjuvant, diluent or carrier; and
(2) a kit of parts comprising components:
(a) a pharmaceutical formulation including a compound of formula I or Ia, as hereinbefore defined, in admixture with a pharmaceutically-acceptable adjuvant, diluent or carrier; and
(b) a pharmaceutical formulation including a platin in admixture with a pharmaceutically-acceptable adjuvant, diluent or carrier,
   which components (a) and (b) are each provided in a form that is suitable for administration in conjunction with the other.

In certain embodiments of the ninth aspect of the invention, the combination product comprises:
(A) a first amount of a compound of formula I or Ia as hereinbefore defined, or a pharmaceutically acceptable derivative thereof; and
(B) a second amount of a platin, or a pharmaceutically acceptable derivative thereof,
   wherein each of components (A) and (B) is formulated in admixture with a pharmaceutically-acceptable adjuvant, diluent or carrier and wherein
   (i) the first and second amounts are both (separately) therapeutically effective amounts, or
   (ii) the first and second amounts together comprise a therapeutically effective amount, or
   (iii) the first and second amounts together comprise a synergistically effective amount.

The term “synergistically effective amount”, when used herein, includes references to combined amounts of components (A) and (B) of the combination product that produce a therapeutic effect that could not be predicted (i.e. goes beyond the mere additive effects) based upon the therapeutic effects of the same quantities of components (A) and (B) when administered separately. In this respect, the therapeutic effect may be in relation to diseases that are treatable by inhibition of telomerase activity, regulation of cell proliferation or exhibition of anti-proliferative properties (e.g. diseases characterised by increased cell-proliferation, such as cancer).

As stated above, there may be several advantages of the compounds of the invention. Further, BSG17 has been shown to possess advantageous properties over known telomerase inhibitors e.g. BR-ACO-19 (BSG01). Surprisingly, computer modelling suggest that compounds with an extra carbon atom in the linker group at the 9-position (i.e. making a benzyl substituent) would bind more effectively to the human G-quadruplex molecular structure (Parkinson et al, 2002), than compounds typified by BR-ACO-19. This modification to the linker group would enable the aromatic ring to overlap with a guanine base and so increase the predicted the binding affinity. The compound BSG17 has such a feature, together with two fluorine atoms attached on the 9-position of the benzyl ring, which confer properties in accord with the above desired features.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### DEFINITIONS

The term “carbo,” “carbyl,” “hydrocarbon” and “hydrocarbyl,” as used herein, pertain to compounds and/or groups which have only carbon and hydrogen atoms.

The term “hetero,” as used herein, pertains to compounds and/or groups which have at least one heteroatom, for example, multivalent heteroatoms (which are also suitable as ring heteroatoms) such as boron, silicon, nitrogen, phosphorus, oxygen, and sulphur, and monovalent heteroatoms, such as fluorine, chlorine, bromine, and iodine.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups).

The term “cyclic,” as used herein, pertains to compounds and/or groups which have one ring, or two or more rings (e.g., Spiro, fused, bridged).

The term “ring,” as used herein, pertains to a closed ring of from 3 to 10 covalently linked atoms, more preferably 3 to 8 covalently linked atoms.

The term “aromatic ring,” as used herein, pertains to a closed ring of from 3 to 10 covalently linked atoms, more preferably 5 to 8 covalently linked atoms, which ring is aromatic.

The term “heterocyclic ring,” as used herein, pertains to a closed ring of from 3 to covalently linked atoms, more preferably 3 to 8 covalently linked atoms, wherein at least one of the ring atoms is a multivalent ring heteroatom, for example, nitrogen, phosphorus, silicon, oxygen, and sulphur, though more commonly nitrogen, oxygen, and sulphur.

The term “alicyclic,” as used herein, pertains to compounds and/or groups which have one ring, or two or more rings (e.g., spiro, fused, bridged), wherein said ring(s) are not aromatic.

The term “aromatic,” as used herein, pertains to compounds and/or groups which have one ring, or two or more rings (e.g., fused), wherein said ring(s) are aromatic.

The term “heterocyclic,” as used herein, pertains to cyclic compounds and/or groups which have one heterocyclic ring, or two or more heterocyclic rings (e.g., spiro, fused, bridged), wherein said ring(s) may be alicyclic or aromatic.

The term “heteroaromatic,” as used herein, pertains to cyclic compounds and/or groups which have one heterocyclic ring, or two or more heterocyclic rings (e.g., fused), wherein said ring(s) is aromatic.

For convenience, certain chemical moieties are represented herein using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, certain chemical compounds are represented herein using well-known abbreviations, including but not limited to, methanol (MeOH), ethanol (EtOH), iso-propanol (iPrOH), methyl ethyl ketone (MEK), ether or diethyl ether (Et2O), acetic acid (AcOH), dichloromethane (methylene chloride, DCM), trifluoroacetic acid (TFA), dimethylformamide (DMF), 4-(dimethylamino)pyridine (DMAP), tetrahydrofuran (THF), and dimethylsulphoxide (DMSO).

The phrase “optionally substituted,” as used herein, pertains to a parent group which may be unsubstituted or which may be substituted

Unless otherwise specified, the term “substituted,” as used herein, pertains to a parent group which bears one or more substituents. The term “substituent” is used herein in the conventional sense and refers to a chemical moiety which is covalently attached to, appended to, or if appropriate, fused to, a parent group.

The term “chemically protected form,” as used herein, pertains to a compound in which one or more reactive functional groups are protected from undesirable chemical reactions, that is, are in the form of a protected or protecting group (also known as a masked or masking group or a blocked or blocking group).

The term “prodrug,” as used herein, pertains to a compound which, when metabolised (e.g., in vivo), yields the desired active compound.

The term “active,” as used herein, pertains to compounds which are capable of inhibiting telomerase and/or of regulating cell proliferation.

The terms “cell proliferation,” “proliferative condition,” “proliferative disorder,” and “proliferative disease,” are used interchangeably herein and pertain to an unwanted or uncontrolled cellular proliferation of excessive or abnormal cells which is undesired, such as, neoplastic or hyperplastic growth, whether in vitro or in vivo.

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included. The term “treatment” also includes combination treatments and therapies, in which two or more treatments or therapies are combined, for example, sequentially or simultaneously.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio. The therapeutic effect may be objective (i.e. measurable by some test or marker) or subjective (i.e. the subject gives an indication of or feels an effect).

The term “anti-proliferative agent” as used herein, pertains to a compound which treats a proliferative condition (i.e., a compound which is useful in the treatment of a proliferative condition).

The term “anticancer agent” as used herein, pertains to a compound which treats a cancer (i.e., a compound which is useful in the treatment of a cancer).

### PREFERRED EMBODIMENTS

Examples embodying certain preferred aspects of the invention will now be described with reference to the following figures in which:—

FIG. 1—Synthetic route to, and structure of, BSG17

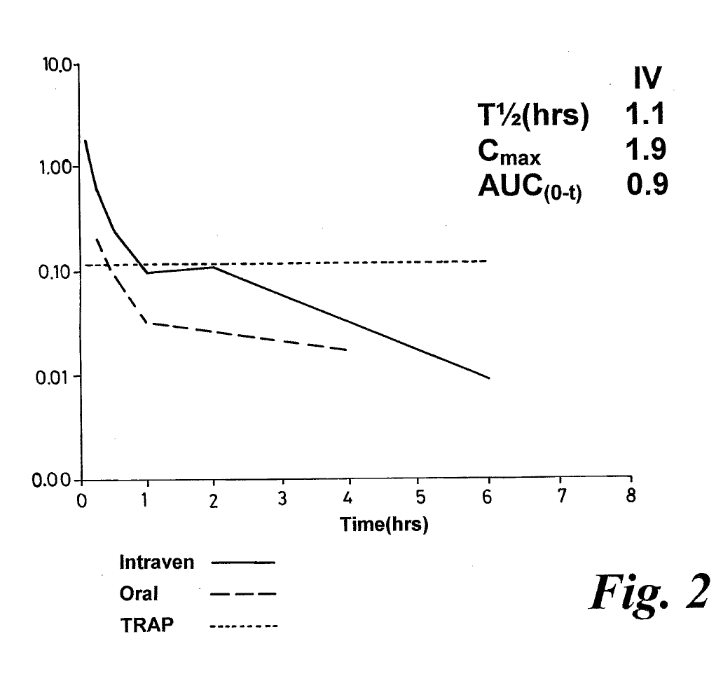

FIG. 2—Plasma Kinetics of BSG01—T_{1/2} (hrs=1.1; Cₘₐₓ (μM)=1.9 and AUC(0-t)=0.96

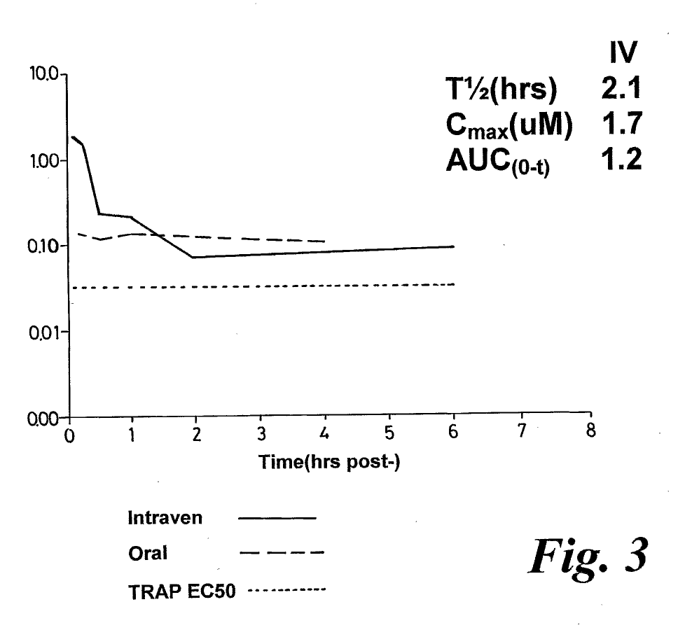

FIG. 3—Plasma Kinetics of BSG17—T_{1/2} (hrs=2.1; Cₘₐₓ (μM)=1.7 and AUC(0-t)=1.23

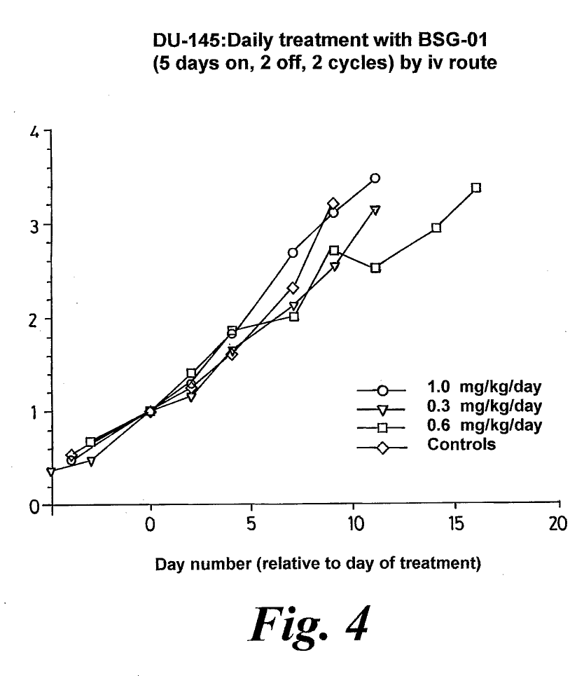

FIG. 4—Xenograft line DU145—Daily treatment of BSG-01 (5 days on, 2 off, 2 cycles) by iv route with amounts 0.3 mg/kg.day, 0.6 mg/kg/day and 1.0 mg/kg.day showing relative tumour volume.

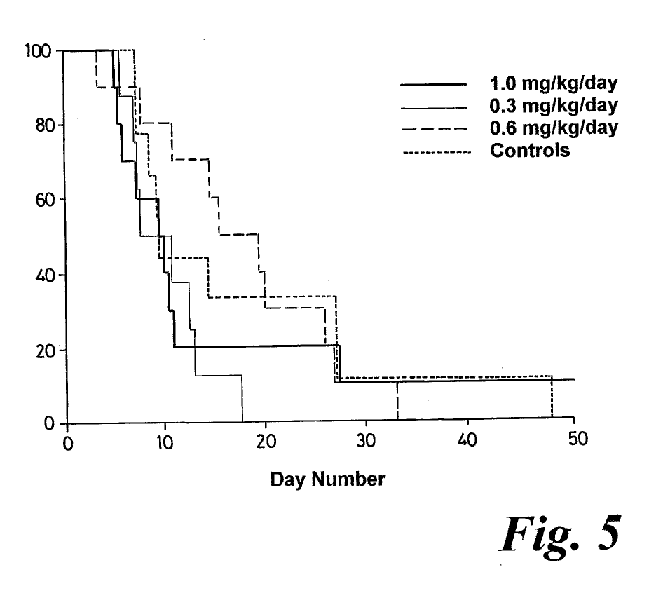

FIG. 5—BSG01 in established DU145 xenografts (iv route) (5 days on, 2 off, 2 cycles) by iv route with amounts 0.3 mg/kg.day, 0.6 mg/kg/day and 1.0 mg/kg.day showing % mice<3V.

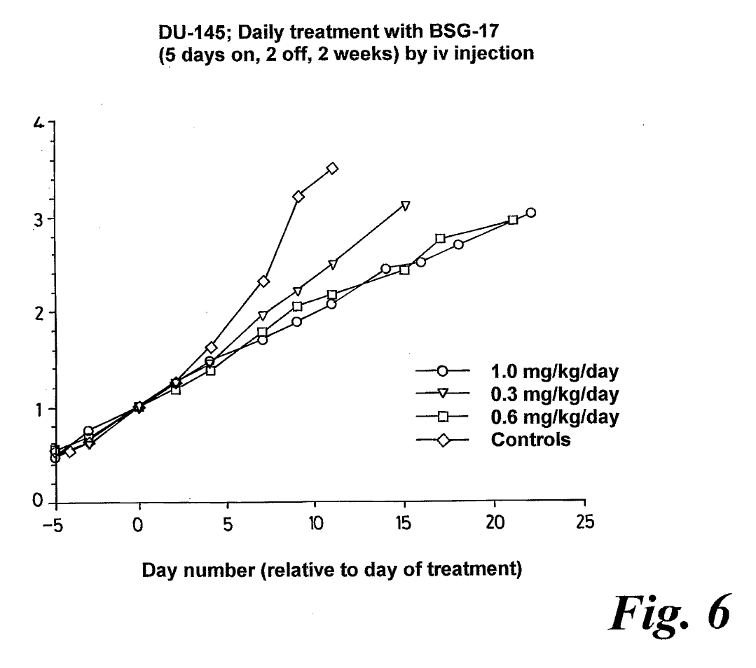

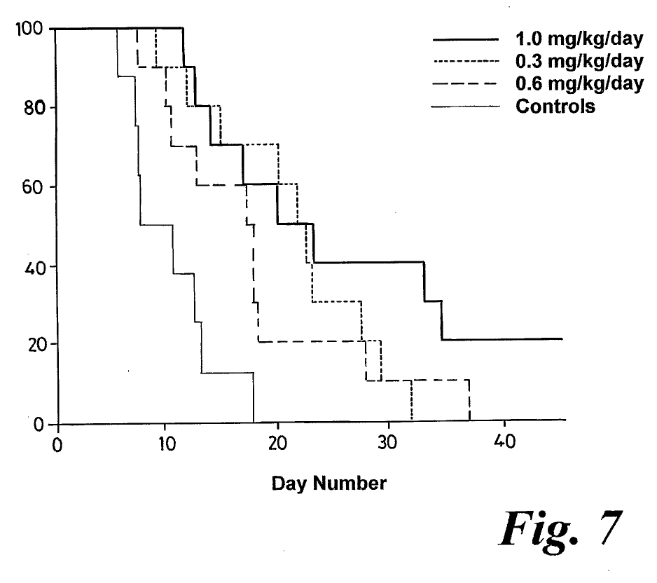

FIG. 6—Xenograft line DU145—Daily treatment of BSG-17 (5 days on, 2 off, 2 cycles) by iv route with amounts 0.3 mg/kg.day, 0.6 mg/kg/day and 11.0 mg/kg.day showing relative tumour volume.

FIG. 7—BSG17 in established DU145 xenografts (iv route) (5 days on, 2 off, 2 cycles) by iv route with amounts 0.3 mg/kg.day, 0.6 mg/kg/day and 1.0 mg/kg.day showing % mice<3V.

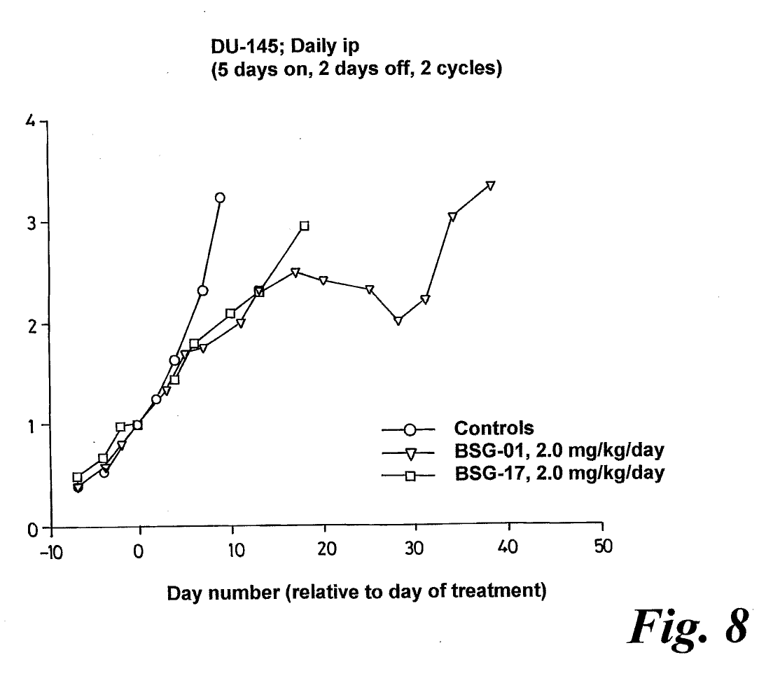

FIG. 8—DU145 xenografts, daily ip treatment with both BSG01 and BSG17 showing % tumour volume.

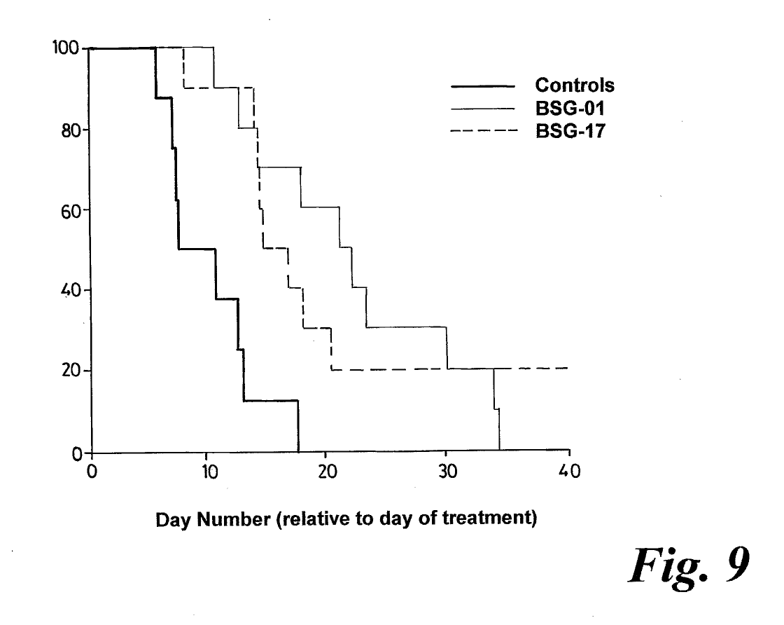

FIG. 9—DU145 xenografts daily ip treatment with both BSG01 and BSG17 showing % mice<3V.

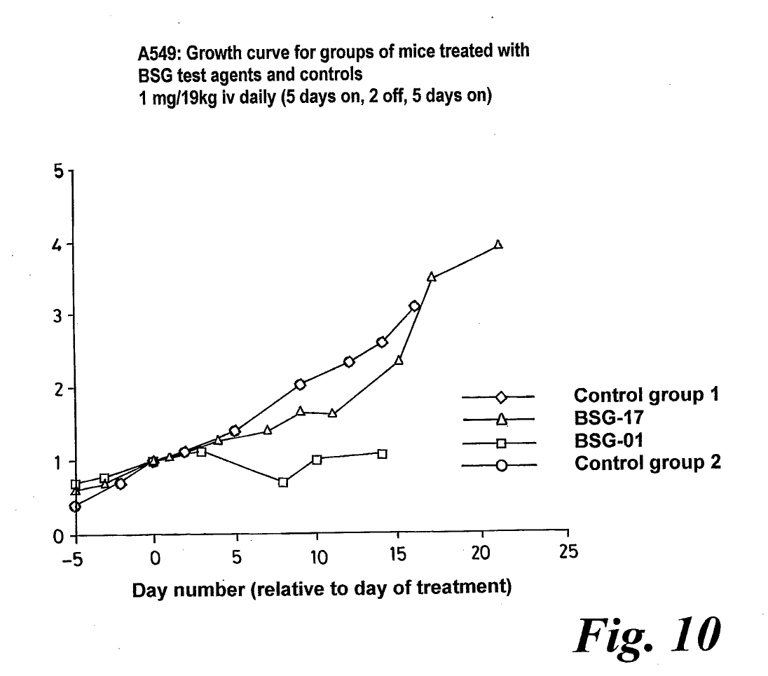

FIG. 10—A549 xenograft model—comparison of BSG01 and BSG17 with controls. (5 days on, 2 off, 5 days on) showing % tumour volume.

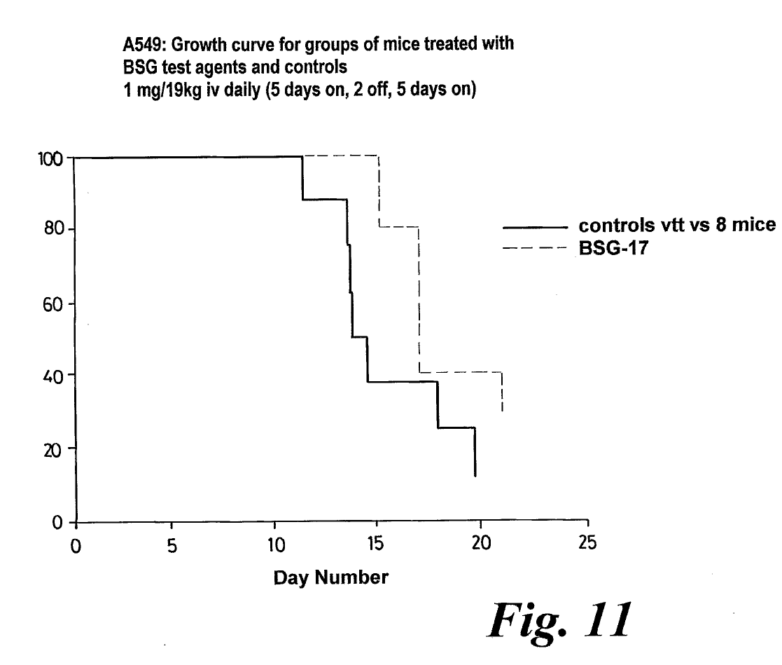

FIG. 11—A549 xenograft model—comparison of BSG17 with controls. (5 days on, 2 off, 5 days on)showing % mice<3V.

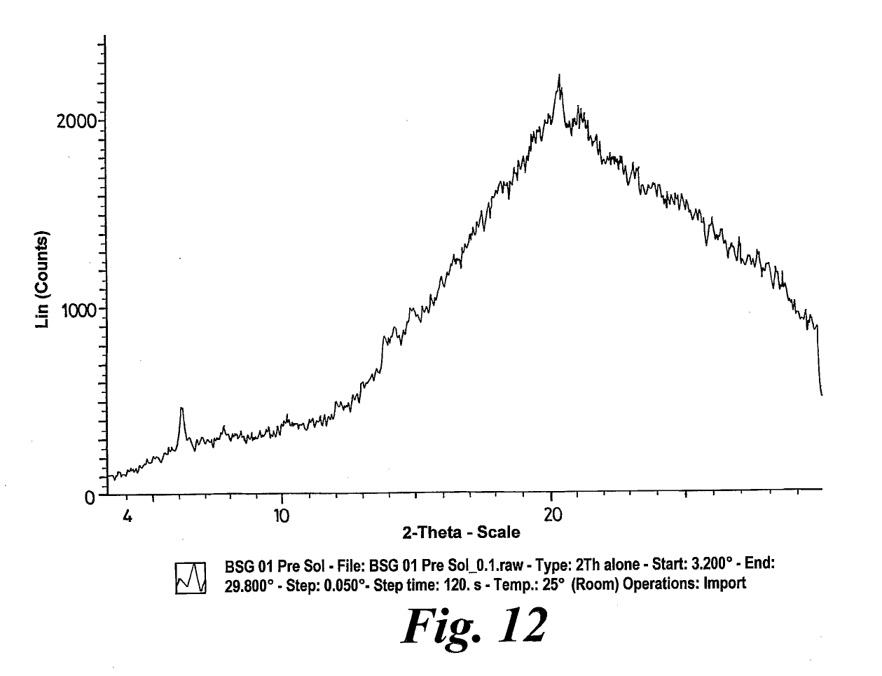

FIG. 12—BSG01 XRPD pattern on material pre-solubilisation.

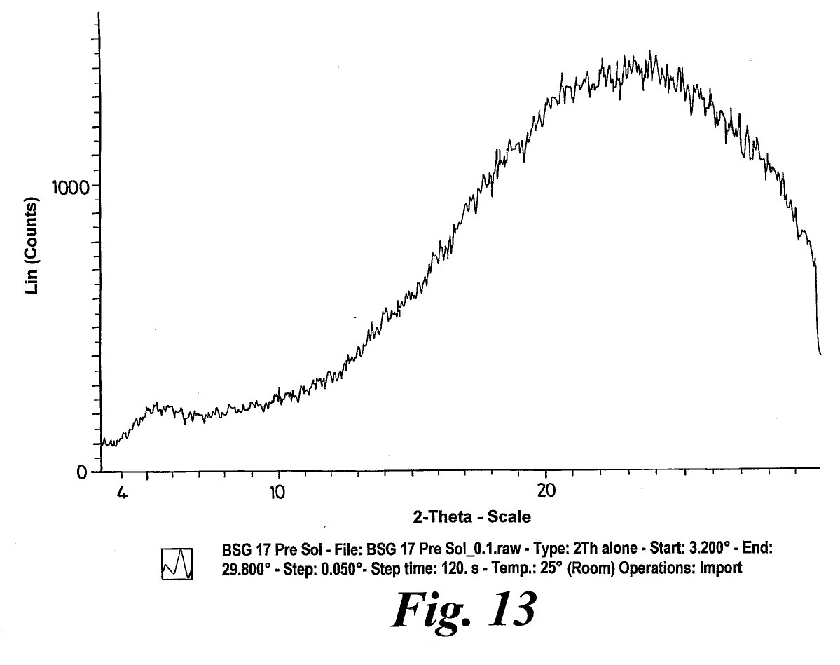

FIG. 13—BSG17 XRPD pattern on material pre-solubilisation.

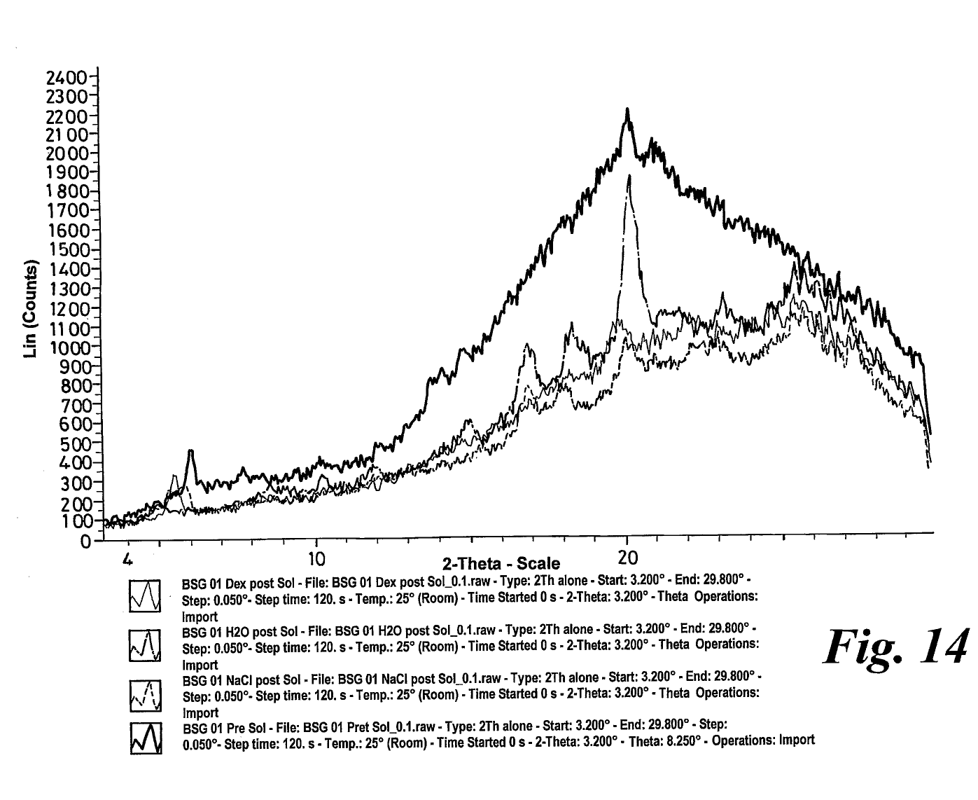

FIG. 14—BSG01 XRPD patterns of recovered solid from solubility assay in Water, 0.9% NaCl and 5% Dextrose.

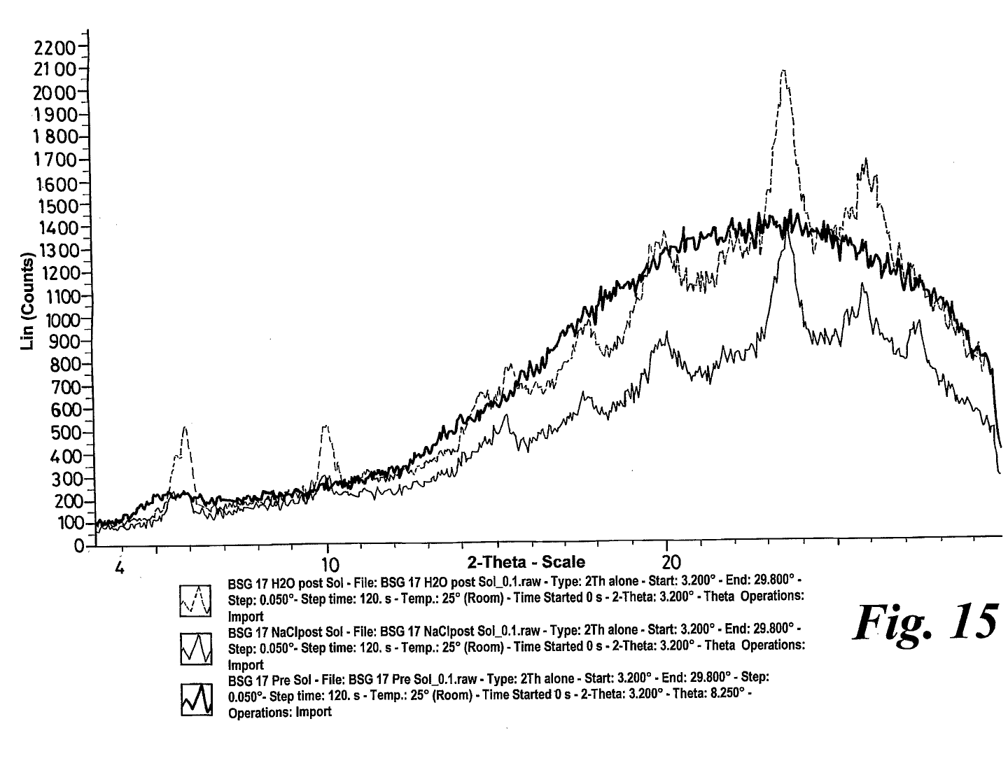

FIG. 15—BSG17 XRPD patterns of recovered solid from solubility assay in Water, 0.9% NaCl and 5% Dextrose.

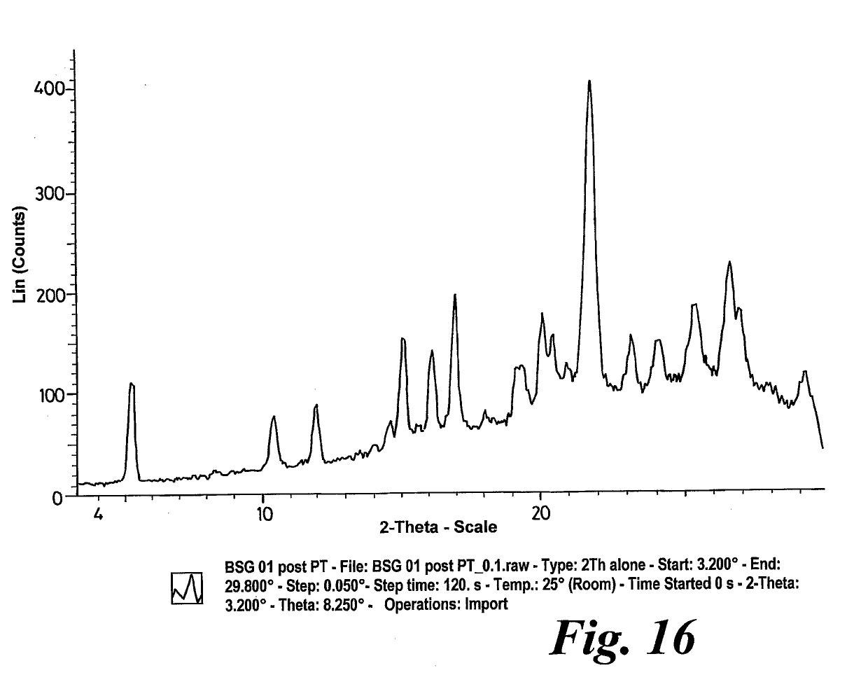

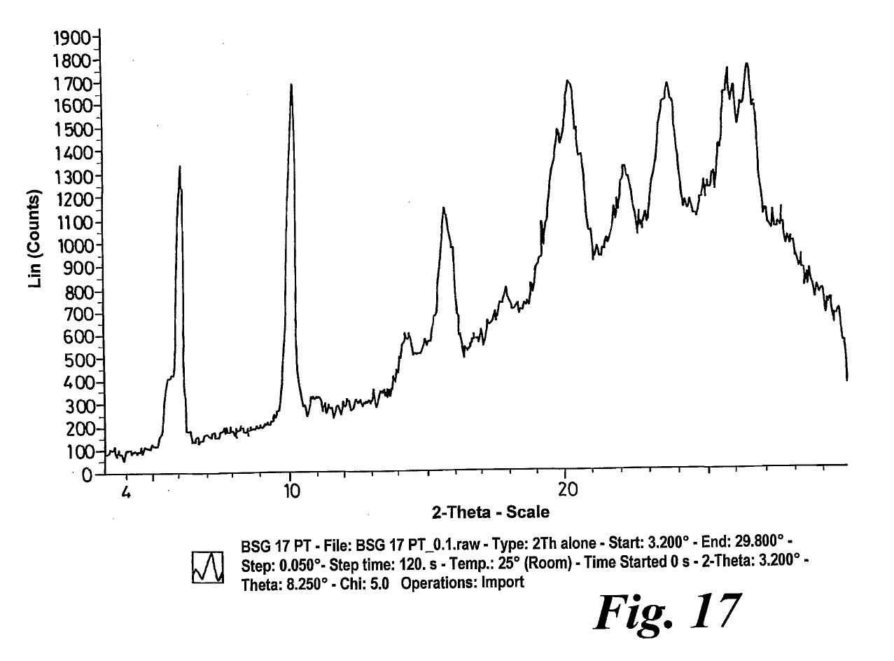

FIG. 16—BSG01 XRPD pattern of recovered solid from potentiometric titration in MeOH/Water/0.15M KCl and at approx pH11.2

FIG. 17—BSG17 XRPD pattern of recovered solid from potentiometric titration in MeOH/Water/0.15M KCl and at approx pH11.2

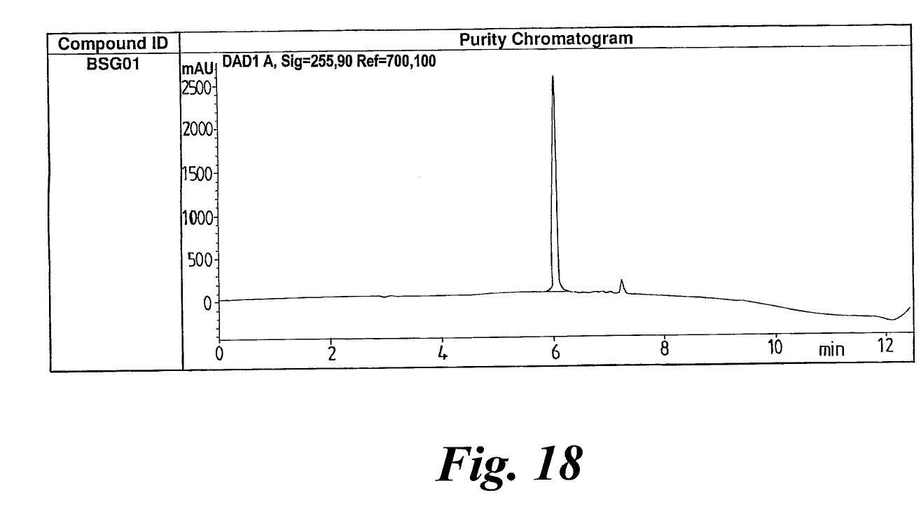

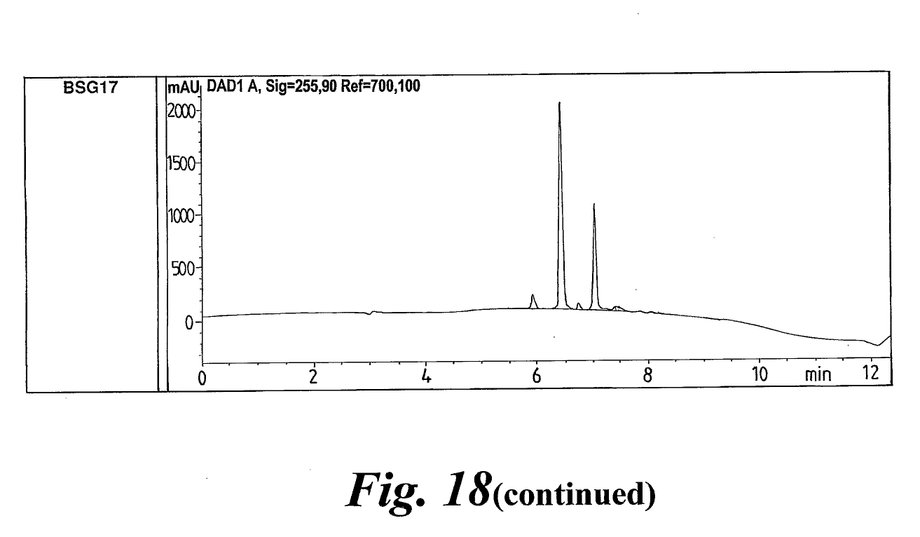

FIG. 18—Chromatograms for Purity Analysis—BSG01 and BSG17

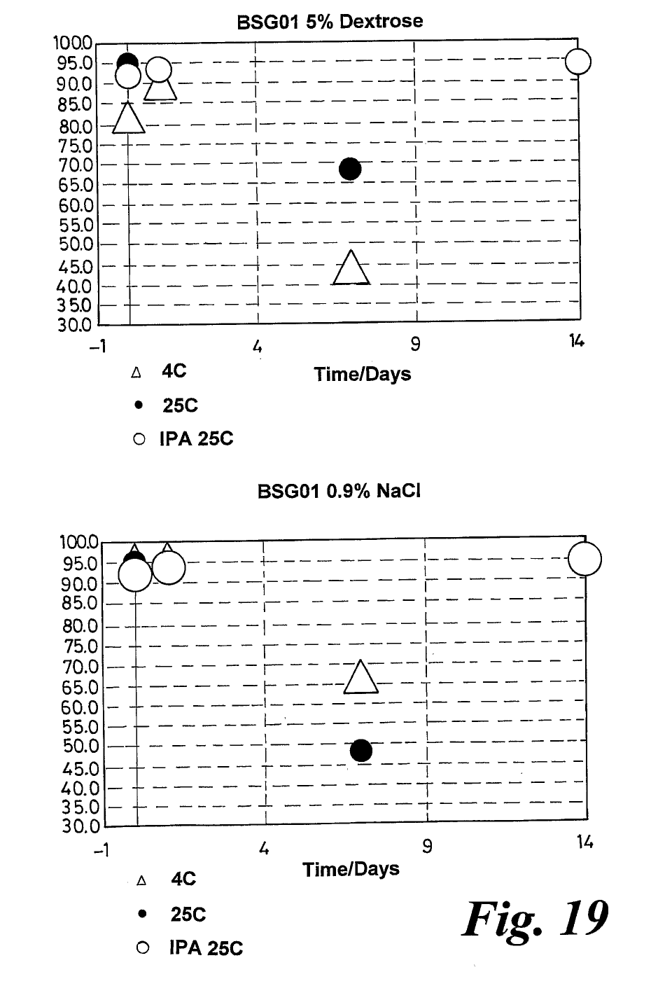

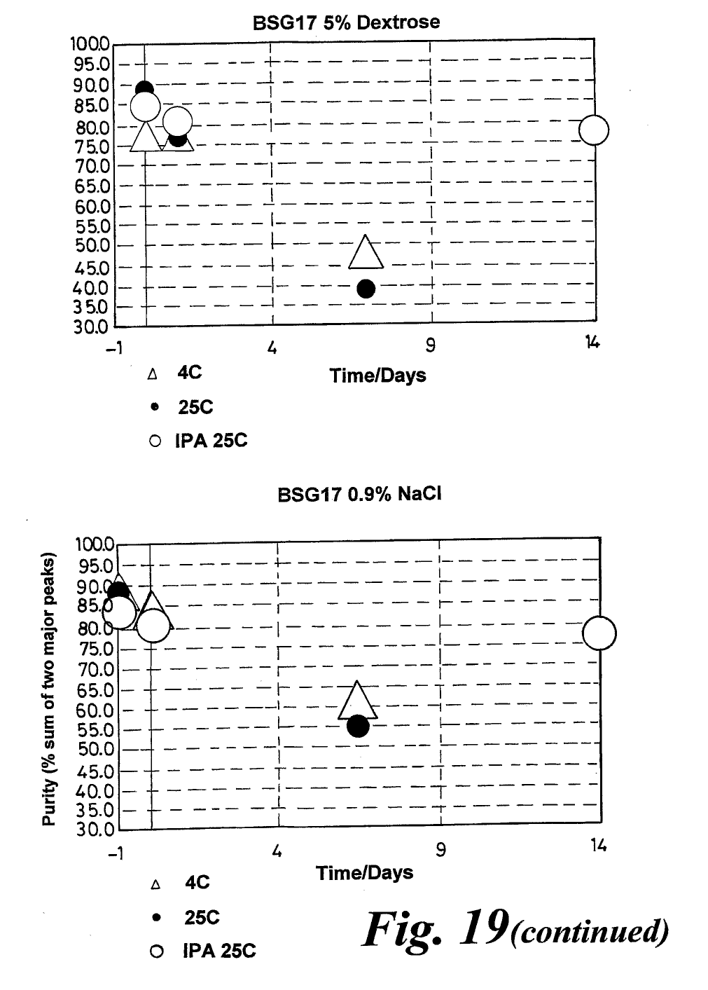

FIG. 19—Purity Degradation—BSG01 and BSG17 in both 5% Dextrose and 0.9% NaCl

FIG. 20—BSG-17, BSG-18-22, BSG-24 and BSG-43 to BSG-49

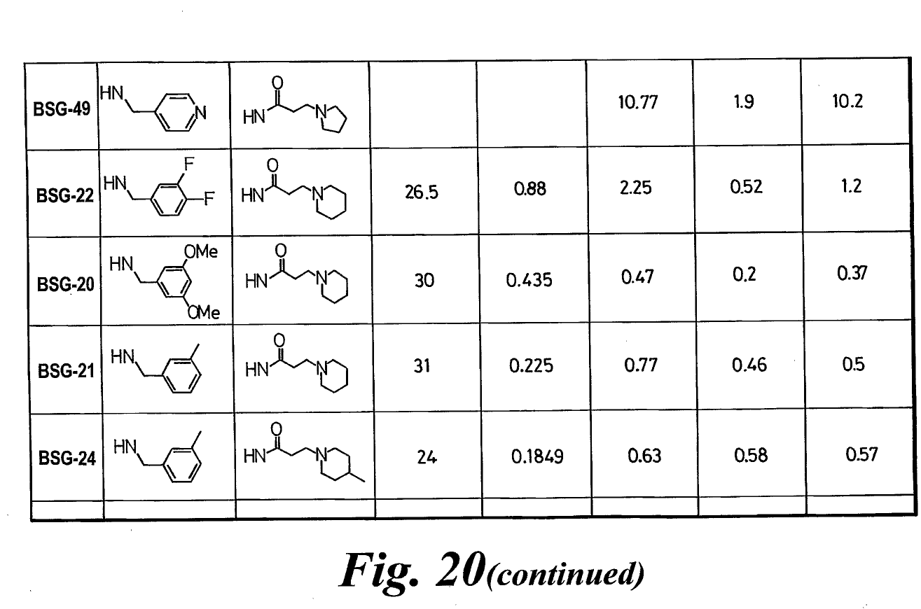

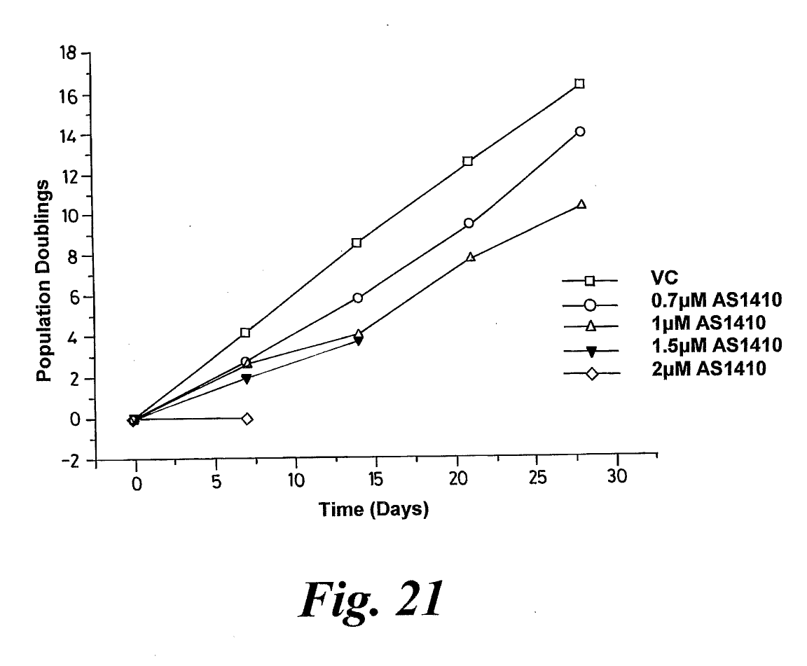

FIG. 21—The effect of AS1410 (alone) on the growth of MCF7 cells in vitro.

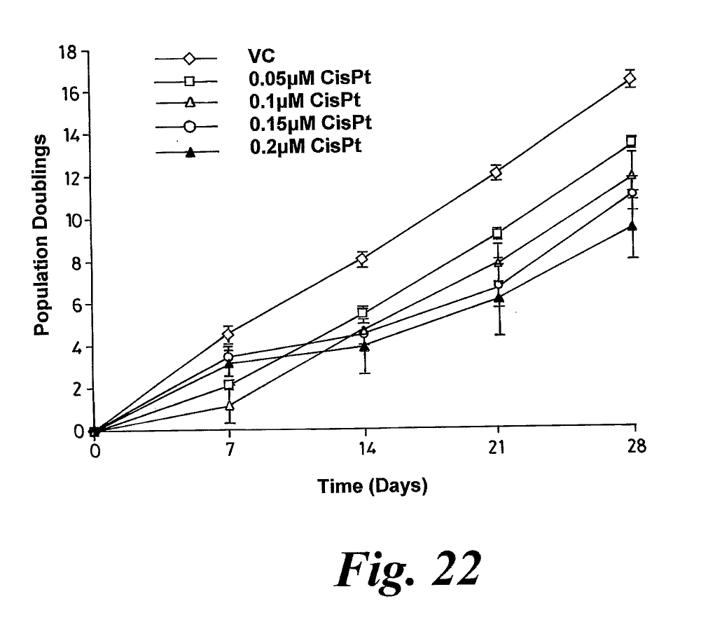

FIG. 22—The effect of cisplatin (alone) on the growth of MCF7 cells in vitro.

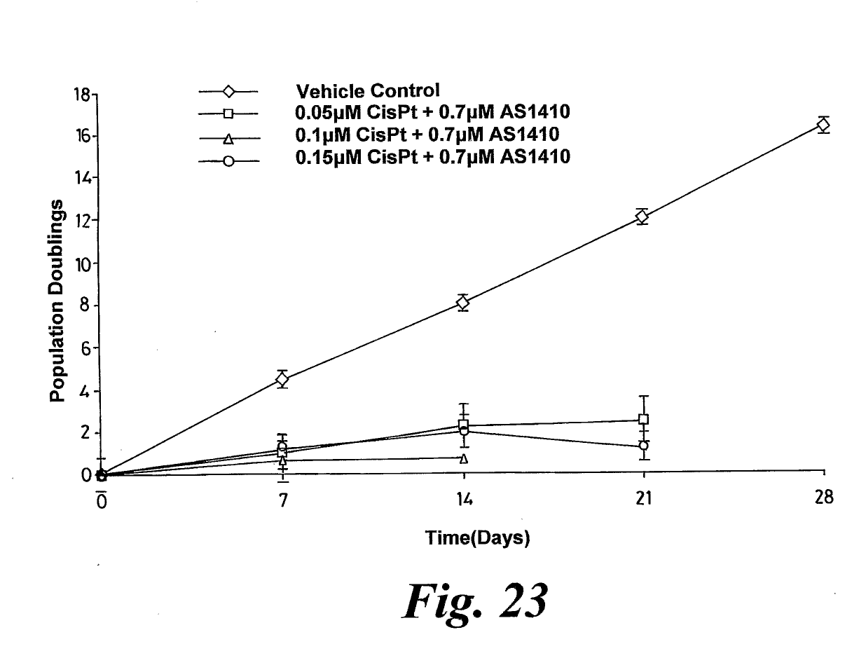

FIG. 23—The effect of the combination of AS1410 and cisplatin on the growth of MCF7 cells in vitro.

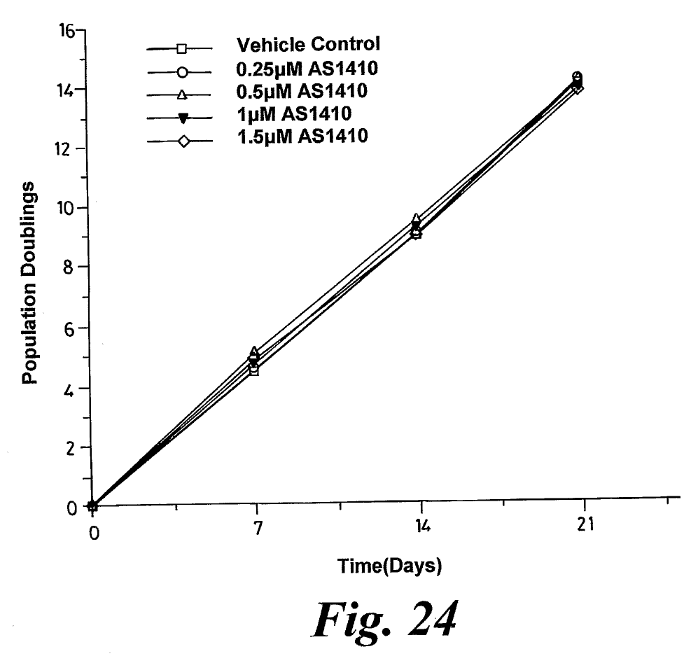

FIG. 24—The effect of AS1410 (alone) on the growth of A549 cells in vitro.

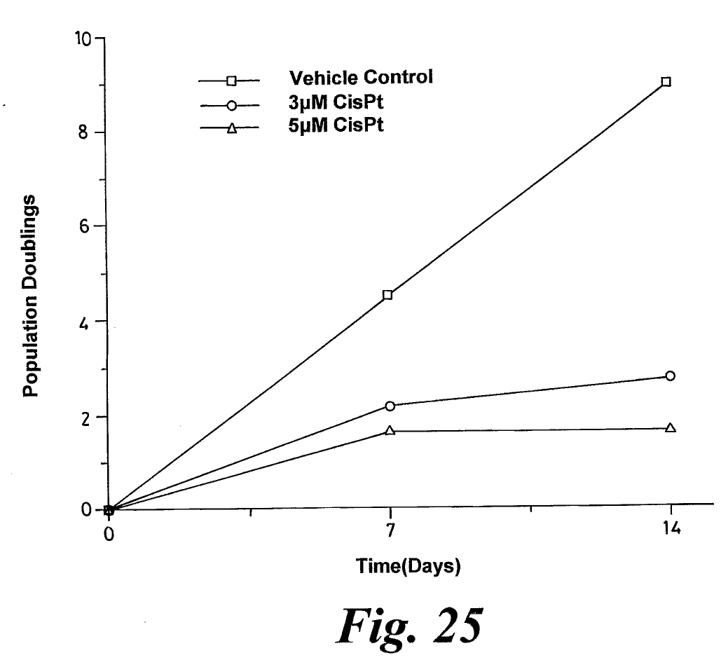

FIG. 25—The effect of cisplatin (alone) on the growth of A549 cells in vitro.

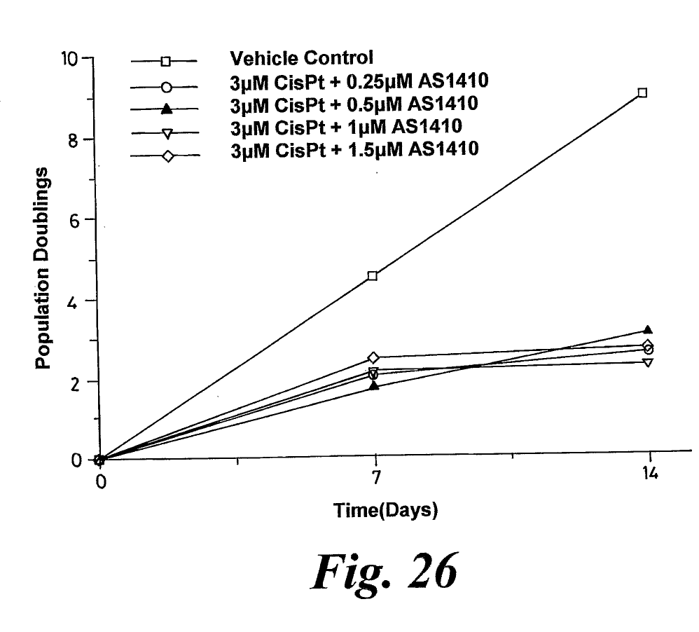

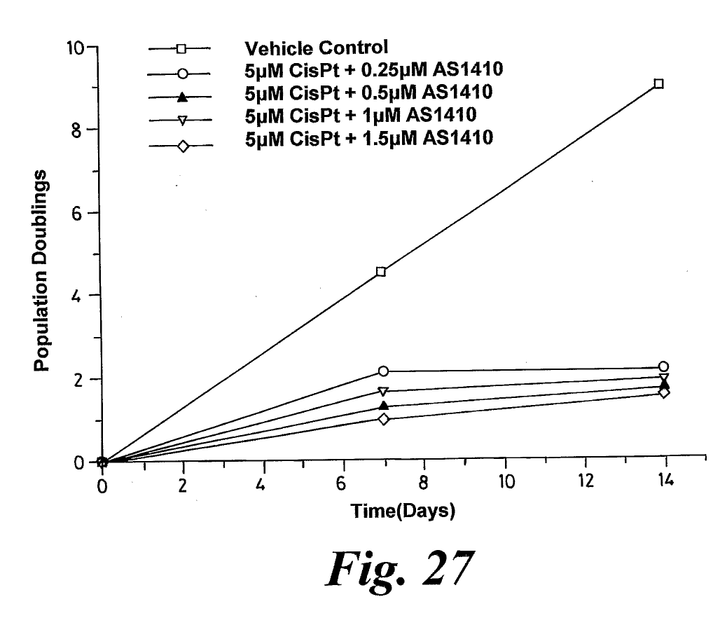

FIGS. 26 and 27—The effect of the combination of AS1410 and cisplatin on the growth of A549 cells in vitro.

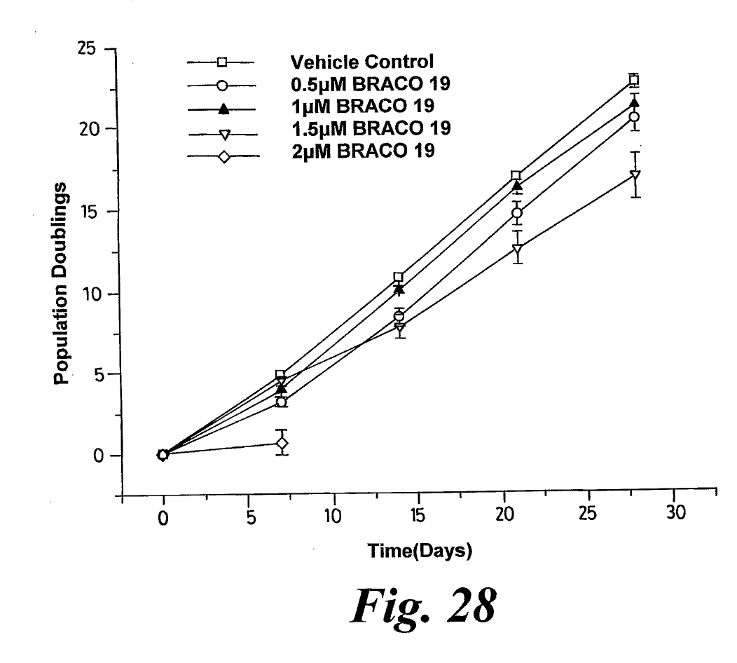

FIG. 28—The effect of BRACO19 (i.e. BSG-01) (alone) on the growth of A431 cells in vitro.

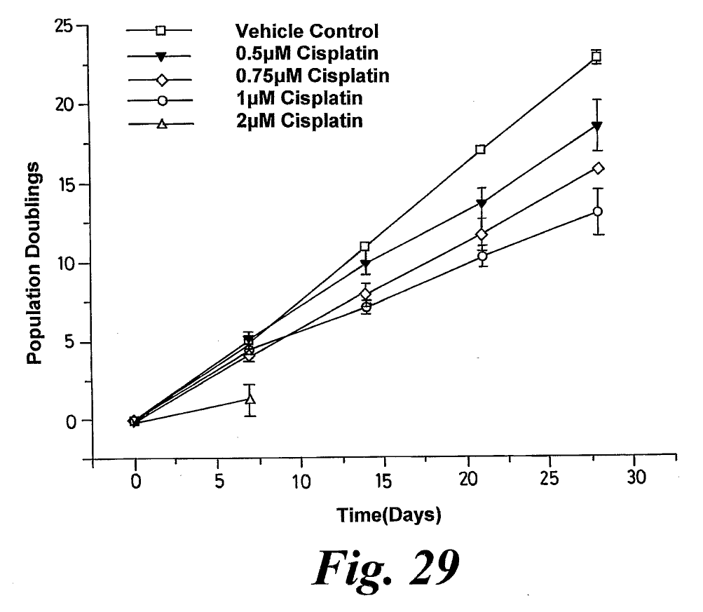

FIG. 29—The effect of cisplatin (alone) on the growth of A431 cells in vitro.

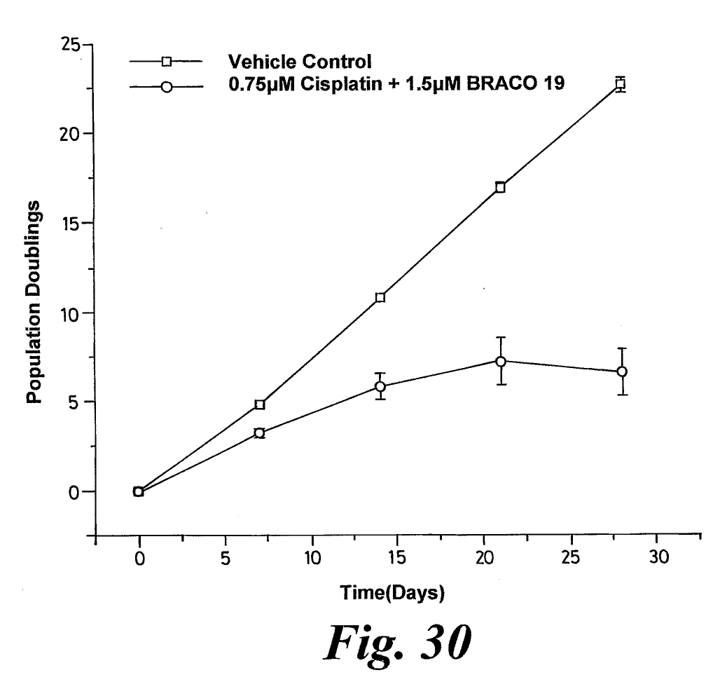

FIG. 30—The effect of the combination of BRACO19 (i.e. BSG-01) and cisplatin on the growth of A431 cells in vitro.

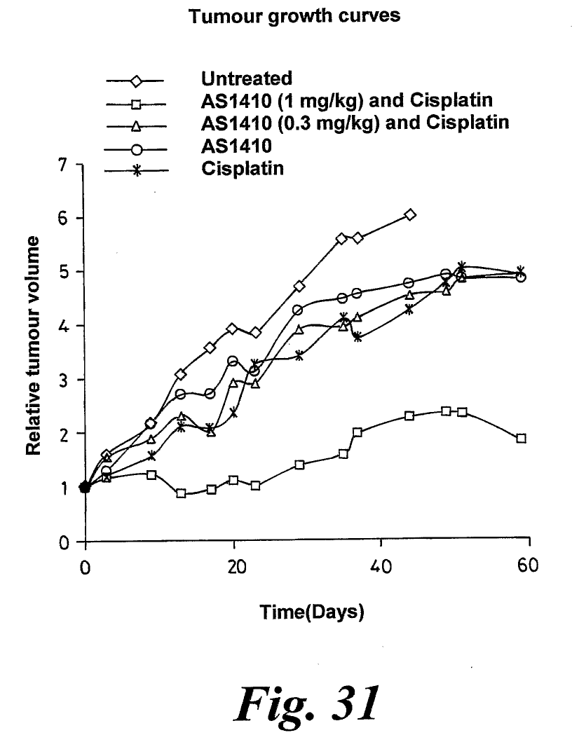

FIG. 31—A549 xenograft model—Comparison of tumour growth curves for AS1410 (alone), ciplatin (alone) and the combination.

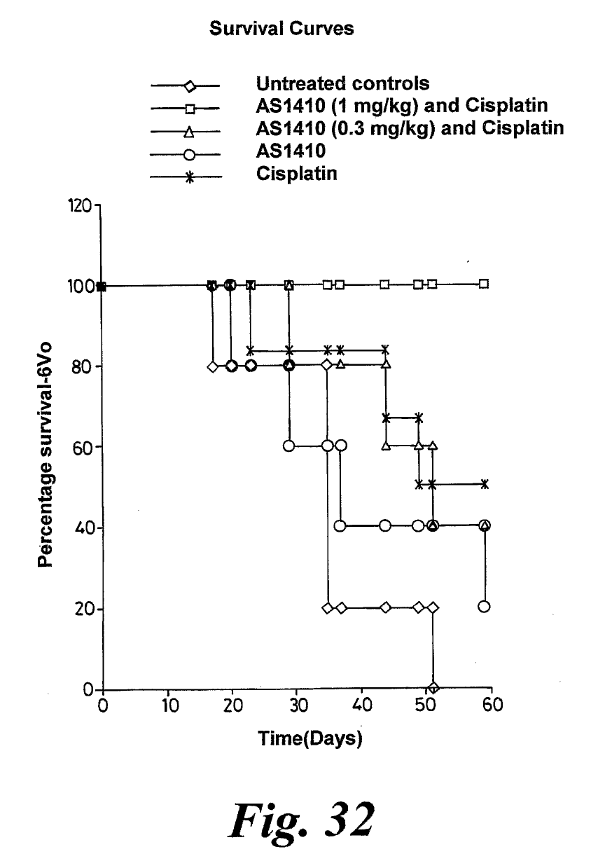

FIG. 32—A549 xenograft model—Comparison of survival curves for AS1410 (alone), ciplatin (alone) and the combination

Various comparative studies were performed, comparing BSG-17 (also referred to herein as AS1410) with BSG-01 (also referred to as BRACO19). For the avoidance of doubt, the these compounds have the following structures:

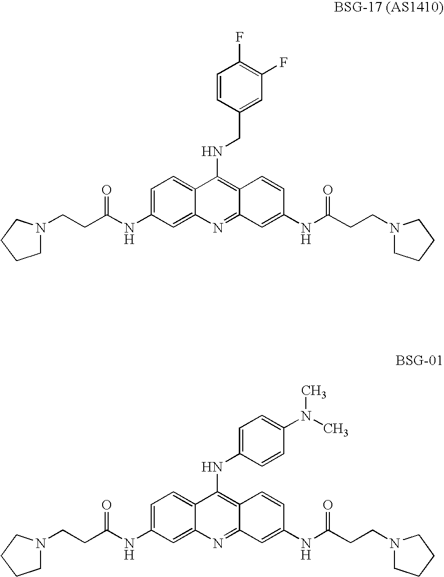

### EXAMPLES

### Example 1

### Compound Synthesis

The acridone and acridine compounds of the present invention may be prepared, for example, by the methods illustrated in FIG. 1, or other well known methods such as Matsumura, 1929 and Korolev et al., 1977 (and references cited therein) or by adaptation of any of these methods in ways within the knowledge of the skilled person.

### Example 2

### Biological Data

### Taq Polymerase Assay

All compounds were tested using a Taq assay to eliminate broad-spectrum polymerase inhibitors and thus filter out any false positives which might have occurred in the TRAP assay. Thus, preferred compounds are “Taq-negative.” Compounds were tested as their acid addition salts at various final concentrations (0.1, 0.5, 1, 5, 10, 20 and 50 μM) in a PCR 50 μL master mix containing 10 ng pCl-neo mammalian expression vector (Promega, Southampton, UK) and forward (GGAGTTCCGCGTTACATAAC) and reverse (GTCTGCTCGAAGCATTAACC) primers (200 nmol) as described in the art (see, e.g., Perry et al., 1998a). The product of approximately 1 kb was visualised on a 2% w/w agarose gel following amplification (30 cycles of 94° C. for 1 min, 55° C. for 1 min and 72° C. for 2.5 min). The Taq assay was carried out until no Taq polymerase inhibition was observed. All compounds were found to be Taq negative.

### Modified Telomeric Repeat Amplification Protocol (TRAP) Assay

The ability of compounds to inhibit telomerase in a cell-free assay was assessed with a modified TRAP assay using extracts from exponentially growing A2780 human ovarian carcinoma cells. The TRAP assay was performed in 2 steps:
(a) telomerase-mediated extension of the forward primer (TS: 5′-AATCCGTCGAGCAGAGTT, Oswel Ltd., Southampton, UK) contained in a 40 μL reaction mix comprising TRAP buffer (20 mM Tris-HCl (pH 8.3), 68 mM KCl, 1.5 mM MgCl2, 1 mM EGTA, 0.05% v/v Tween 20), 0.05 μg bovine serum albumin, 50 μM of each deoxynucleotide triphosphate, 0.1 μg TS primer, and 3 μCi of [α-P]dCTP (Amersham plc, UK)Protein (40 ng or 20 ng) was then incubated with the reaction mix and agent (acid addition and quaternary dimethiodide salts) at final concentrations of up to 50 μM for 20 min at 25° C. A lysis buffer (no protein) control, heat-inactivated protein control, and 50% protein (20 ng or 10 ng) control were included in each assay; and
(b) while heating at 80° C. in a PCR block of a thermal cycler (Hybaid, UK) for 5 min to inactivate telomerase activity, 0.1 μg of reverse CX primer (3′-AATCCCATTCCCATTCCCATTCCC-5′) and 2 Units of Taq DNA polymerase (“red hot”, Advanced Biotechnologies) were added. A 3-step PCR was then performed: 94° C. for 30 s, 50° C. for 30 s, and 72° C. for 1 min for 31 cycles. Telomerase-extended PCR products in the presence or absence of compounds were then determined either by electrophoretic separation using 8% w/w acrylamide denaturing gels and analysis by phosphorimaging or autoradiography, or by harvesting on Whatman filters (25 mm glass microfibre) and analysis by liquid scintillation counting.

### FRET DNA Melting Assay

The ability of the compounds to stabilise G-quadruplex DNA was investigated using a fluorescence resonance energy transfer (FRET) assay modified to be used as a high-throughput screen in a 96-well format. The labelled oligonucleotide F21T (5′-FAM-dGGG(TTAGGG)-3-TAMRA-3′) used as the FRET probe was diluted from stock to the correct concentration (400 nM) in a 50 mM potassium cacodylate buffer (pH 7.4) and then annealed by heating to 85° C. for 10 min., followed by cooling to room temperature in the heating block.

Compounds were stored as 10 mM stock solutions in DMSO; final solutions (at 2× concentration) were prepared using water or 1M HCl in the initial 1:10 dilution, after which 50 mM potassium cacodylate buffer (pH 7.4) was used in all subsequent steps. 96-well plates (MJ Research, Waltham, Mass.) were prepared by aliquoting 50 μl of the annealed DNA to each well, followed by 5041 of the compound solutions. Measurements were made on a DNA Engine Opticon (MJ Research) with excitation at 450-495 nm and detection at 515-545 nm. Fluorescence readings were taken at intervals of 0.5° C. over the range 30-100° C., with a constant temperature being maintained for 30 seconds prior to each reading to ensure a stable value. Final analysis of the data was carried out using a script written in the program Origin 7.0 (OriginLab Corp., Northampton, Mass.). The advanced curve-fitting function in Origin7.0 was also used to obtain the relevant curves for the calculation of [conc]_{ΔTm=20} values, as described in Guyen et al., 2004.

### Growth Inhibition Assay

Growth inhibition was measured in three human ovarian carcinoma cell lines (A2780, CHI, and SKOV-3) and one human cervix carcinoma cell line (A431) using the sulphorhodamine B (SRB) assay. Briefly, between 3000 and 6000 cells were seeded into the wells of 96-well microtitre plates and allowed to attach overnight. Compounds (acid addition and quaternary dimethiodide salts) were dissolved at 500 μM in water and immediately added to wells in quadruplicate at final concentrations of 0.05, 0.25, 1, 5 and 25 μM. Following an incubation period of 96 hr, remaining cells were fixed with ice-cold 10% w/v trichloroacetic acid (30 min) and stained with 0.4% SRB in 1% v/v acetic acid (15 min) Mean absorbance at 540 nm for each drug concentration was expressed as a percentage of the control untreated well absorbance, and IC50 values (concentration required to inhibit cell growth by 50%) were determined for each.

### Results

TABLE 1
Metabolic stability results

| | | | Cytosol | Cytosol |
|---|---|---|---|---|
| | HLM (t = 45) | HLM (t = 90) | (t = 30) | (t = 60) |
| BSG01 | 6.1 | 11.2 | 3.5 | 6.3 |
| BSG17 | 3.1 | 12.4 | ND | ND |

### Example 4

### Pharmacokinetic Evaluation

### Test compound

The test article is identified as BSG17 and has been studied in comparison to BSG01 (BRACO-19). The vials containing bulk test article were stored at room temperature (15-30° C.).

### Test Formulation Preparation

The test compound was reconstituted in 0.9% physiological saline B.P. to the required final concentrations. To ensure the stability of the test formulations, prepared formulations were administered within two hours of preparation.

### Test Compound Administration

Treated mice received a single bolus intravenous injection or oral gavage of the test formulations at a dose-volume of 10 mL/g bodyweight. Control mice were untreated.

The intravenous and oral routes of administration were selected, as they are potential human therapeutic routes.

### Mice Allocation

The mouse was selected because it is a rodent species and is acceptable to the regulatory authorities for pharmacokinetic studies. Dose levels of 1 mg/kg (intravenous) and 20 mg/kg (oral) were selected for this study.

Female MF1 mice in the weight rang of 26-30 g (6 to 8 weeks of age) were obtained from the BRF Facility, SGHMS/Harlan UK, Bicester and were individually identified using a permanent tail mark (using a non-toxic marker pen). The test article was administered to thirty-six (36) mice after a period of seven (7) days of acclimatisation to the study room.

### Animal Welfare

All procedures in this experiment conformed to the requirements of the Animals (Scientific procedures) Act 1986 and appropriate legislation and guidance documents relating to animal welfare.

Animals were housed in solid bottom cages that comply with the requirements of the Code of Practice for the housing and care of animals used in scientific procedures. A commercially available rodent diet and sterile water was provided ad libitum throughout the study. Wood chips or shavings were provided as bedding for solid-bottom cages.

### Sampling and Analysis Procedures

### Blood Sampling

Blood samples were obtained from three mice per time point and at the time points as specified below:

TABLE 2

| | |
|---|---|
| Intravenous | 5, 15 and 30 minutes, 1, 2, 4 and 8 hours after dosing |
| administration | |
| Oral | 15 and 30 minutes and 1, 2, 4, 8 and 24 hours |
| administration | |

Blood samples were collected by cardiac puncture with mice held under deep halothane anaesthesia. After sampling mice were euthanased by cervical dislocation.

Samples were collected into pre-heparinised syringes and the samples transferred into 1 ml eppendorf tubes. Plasma was prepared by centrifugation, transferred to clean, labelled eppendorf tubes and frozen at approximately −20° C. pending analysis.

Samples were analysed using HPLC or GC-MS/MS methodology.

### Urine Sampling

At the time of euthanasia, urine samples were collected, where possible, by manual palpation of the bladder. Samples were collected into labelled eppendorf tubes and frozen at approximately −20° C. pending analysis.

Samples were analysed using a GC-MS/MS methodology for BRACO-19 (parent) and qualitative identification of metabolites. Prior to analysis, samples for each group/route of administration were pooled as follows:
Pool 1—urine collected up to 1 hr after dosing
Pool 2—urine collected between 2 and 4 hrs after dosing
Pool 3—urine collected between 8 and 24 hrs after dosing

### Results

Composite plasma concentration time curves were generated over the period of 6-8 hours after the bolus iv or oral dose was administered. The results are shown in FIGS. 2 and 3.

The pharmacokinetic parameters of BSG01 and BSG17 are:

TABLE 3

| | | AUC₀₋ₜ | | TRAP | Clearance |
|---|---|---|---|---|---|
| | T_{1/2} (hrs) | (μM · hr) | Cₘₐₓ (μM) | EC₅₀ | (L/hr) |
| BSG01 | 1.1 | 0.96 | 1.9 | 0.115 (16.5x) | 0.06 |
| BSG17 | 2.1 | 1.232 | 1.7 | 0.032 (53x) | 0.04 |

### Example 5

### Efficacy Studies

The objective of this study was to determine the response of a human tumour xenograft implanted in athymic mice to the test compound so as to determine the test compound's anti-proliferative effect.

### Route of Administration

The intravenous route of administration was selected, as it is a potential human therapeutic route.

### Species, Strain and Xenograft Model

The mouse was selected because it is a rodent species and is internationally accepted as the species of choice for human xenograft studies. The nu/nu MF1 strain was selected.

The DU145 metastatic prostate model has been selected for this dose-range finding study as the substituted acridine analogues have been demonstrated to have significant cytostatic activity against this cell line during chronic in vitro cytotoxicity assays.

A high dose level of 1 mg/kg given once daily for 10 days was selected for this study. Low and intermediate dose levels of 0.3 and 0.6 mg/kg/day were also selected to aid characterisation of the dose-response profile.

The study was performed in compliance with the spirit of Good Laboratory Practice and was performed in accordance with normal laboratory practice and SOPs was followed.

### Test Compound

The test compound was BSG17 and was tested in comparison to BSG01 (BR-ACO-19). Their biological and physicochemical properties are summarized as follows:

TABLE 4

| | | Telomerase | | Cytotoxic IC50 (μM) vs. | | |
|---|---|---|---|---|---|---|
| Name | Log P | EC50 (μM) | FRET (μM) | DU145 | MCF7 | A549 |
| BSG01 | 1.40 | 0.115 | 0.72 | 2.30 | 2.53 | 2.42 |
| BSG17 | 1.59 | 0.162 | 0.46 | 9.75 | 2.26 | 2.12 |

The test compound was synthesised and purified at the School of Pharmacy, University of London, UK. The vials containing bulk test article were stored at room temperature (15-30° C.).

BSG17 was also tested in comparison with compounds BSG-18 to BSG-22, BSG24 and BSG-43 to BSG-49. Their biological and physicochemical properties are summarized in FIG. 20.

### Test Formulations

The test articles were reconstituted in 0.9% physiological saline B.P. to the required final concentrations; and where necessary, an organic solvent (e.g. DMSO or ethanol) was used to aid solubilisation of some materials.

Prepared formulations were administered within two hours of preparation.

### Test Compound Administration

Treated mice received a bolus intravenous injection of the test formulation once daily for two weeks (week-days only). A dose-volume of 5 ml/g bodyweight was be used. Control mice were untreated.

### Animals

A total of 175 female MF1 mice were obtained from the Biological Research Facility, SGHMS, London. Animals were in a weight range of 26-30 g (6 to 8 weeks of age). Mice were individually identified using a subcutaneously implanted microchip.

After arrival, animals were allowed to acclimatize to the study room for at least 7 days prior to undergoing the first study procedures.

Animals were housed in solid bottom cages that comply with the requirements of the Code of Practice for the housing and care of animals used in scientific procedures. A commercially available rodent diet and sterile water was provided ad libitum throughout the study. Wood chips or shavings were provided as bedding for solid-bottom cages.

All procedures in this experiment conformed to the requirements of the Animals (Scientific procedures) Act 1986 and appropriate legislation and guidance documents relating to animal welfare.

Mice were allocated to groups as follows:

TABLE 5

| | | | Level |
|---|---|---|---|
| Group No. | Test Compound | Dose | (mg/kg/day) |
| 1 | Control | — | 10 |
| 2 | BSG-01 | 0.3 | 10 |
| 3 | BSG-01 | 0.6 | 10 |
| 4 | BSG-01 | 1.0 | 10 |
| 5 | BSG-17 | 0.3 | 10 |
| 6 | BSG-17 | 0.6 | 10 |
| 7 | BSG-17 | 1.0 | 10 |

### Establishment of Xenografts

The xenografts were prepared from the DU145 cell line (ATCC HTB-81). Cells were grown in accordance with established methodology for this cell line to provide 5×10⁶ cells per mouse.

Xenografts were established in the MF1 mice by subcutaneous injection into the right flank of each mouse (5×10⁶ cells in 100 ml phosphate buffered saline).

Animals were checked regularly until visible tumours appeared. Tumours were then measured in accordance with the relevant SOP three times per week and tumour volume calculated using the formula:

Volume=(π÷6000)×(L×W×H)

Measurements commenced approximately one week before the day of treatment (Day 0) when the xenograft reached an approximate volume of 0.05 cm³ (e.g. dimensions of approximately 5×5×4 mm).

Once the tumours were established, mice were allocated into groups of 5 mice using a randomization procedure based on stratified xenograft volume. Any mouse in which the xenograft did not “take” or the tumour volume was outside the specified range (0.05-0.25 cm3) were excluded.

Measurements were captured electronically using the Quantum system and data transferred to an Access database. This was used to analyse data and create a series of four study reports.

Day 0 is defined as the day on which the average tumour volume is between 0.12 to 0.16 cm³. Relative tumour volume on Day 0 is defined as 1.

Each mouse was observed and tumours measured until the xenografts tripled in volume (3Vₒ) compared with volume on the day of treatment (Vₒ). The elapsed period was the volume tripling time (VTT).

Once tripling was confirmed, mice were killed using an approved humane method.

### Experimental Observations

A record was maintained of any signs seen during or after the dose administration procedure. Individual body weights were recorded at the same frequency and on the same occasions as tumour measurements are performed.

### Results

The results in the DU145 cell line are shown in FIGS. 4 to 9 and the A549 results are shown in FIGS. 10 and 11. BSG17 shows a clear dose response and effects at all three dose levels (0.3, 0.6 and 1.0 mg/kg/day) with an increased effect for longer duration than BSG01.

T-C values of 7, 11.6 or 11 days at 0.3, 0.6 or 1 mg/kg/day.

### Example 6

### Combination studies

### In Vitro

(1) FIGS. 21 to 23 show the effect of AS1410 (alone), cisplatin (alone) and the combination on the growth of MCF7 cells in vitro.

(2) FIGS. 24 to 27 show the effect of AS1410 (alone), cisplatin (alone) and the combination on the growth of A549 cells in vitro.

For the A549 cell line the concentration of AS1410 administered was much lower than IC₅₀ values since the aim was to establish the lowest AS1410 concentration required to produce maximum cell kill in combination

(3) FIGS. 28 to 30 show the effect of BRACO19 (i.e. BSG-01) (alone), cisplatin (alone) and the combination on the growth of A431 cells in vitro.

In this combination study, the following methods were used:

### Cell Lines:

Human breast cancer cell line (MCF7), and lung cancer cell line (A549) (purchased from ATCC) were routinely maintained in 75 cm² flasks with Dulbecco's Modified Eagle Medium (DMEM) (Invitrogen Corporation). Medium was supplemented with L-glutamine 2 mM, non-essential amino acids, foetal bovine serum and hydrocortisone. Cells were incubated at 37° C. and 5% CO₂.

### Preparation of Drugs:

### AS1410

10 mM stock solution was prepared from AS1410 succinate salt (MW 896) in sterile distilled water and kept at 4° C. Further dilution to give a 1 mM solution was carried out in sterile water and appropriate volumes were added to cell culture in long term studies.

### CisPlatin

1 mM cisplatin stock solution was prepared by dissolving the crystalline cis-diamminedichlorplatinum(2) powder (Sigma) in 10 ml 0.1% saline solution. The stock solution was wrapped in foil due to its sensitivity to light. The drug was made freshly on the day of use due to its instability in solution and filter sterilised before adding to cell culture.

### Long Term Combination Experiments:

Long term combination studies were set up for each cell line with single agents and in combination.

The long term studies follow a schedule which was repeated every week for the duration of the experiment (4 weeks). The schedule for each of the cell lines is as follows:
1. On day 1, 1×10⁵ cells were seeded in 75 cm² flasks in 10 ml DMEM with appropriate volumes of 1 mM drug stock solution and incubated as described earlier.
2. On day 3 or 4 cells were retreated with fresh medium containing appropriate concentration of drug and continued to incubate as before.
3. On day 7 cells were counted and 1×10⁵ cells were re-plated as before and experiments were continued for four weeks.
   Concentrations of drugs were according to IC₅₀ values.

### Example 7

### Combination Studies; Xenograft Data

### Preparation

AS1410 was reconstituted in 0.9% saline B.P. to the required final concentration (0.331 and 0.0993) mg/ml of AS1410 succinate salt for intravenous administration).

### Experimental Methods

### Cell Culture

The xenografts were prepared from the lung carcinoma A549 (ATCC Number CCL-185). Cells were grown in accordance with standard operating procedures and established methodology for this cell line to provide 5×10⁶ cells per mouse.

### Establishment and Measurement of Xenografts

A549-derived xenografts were established in 50 female nu/nu MF1 mice respectively by subcutaneous injection into the right flank of each mouse (5×10⁶ cells in 100 μl phosphate buffered saline).

Animals were checked regularly until visible tumours appeared. Tumours were then measured in accordance with the relevant SOP regularly and tumour volume calculated using the formula:

Volume=(π÷6000)×(L×W×H)

Once the tumours were established, the mice were allocated into groups of 9 mice using a randomization procedure based on stratified xenograft volume. Any mouse in which the xenograft did not “take” or the tumour volume was outside the specified range (0.04-0.1 cm³) was excluded.

Measurements were recorded electronically using electronic callipers and the Quantum system for data capture.

Day 0 is defined as the day on which the average tumour volume is between 0.04 to 0.1 cm³. Relative tumour volume on Day 0 is defined as 1.

Each mouse was observed and tumours measured until the xenografts tripled (3V₀) in volume compared with volume on the day of treatment (V₀). The elapsed period is the volume tripling time (VTT).

### Test Article Administration

AS1410 was given by slow bolus intravenous injection. For AS1410 a dose volume 5 μl/g bodyweight was used. Control mice were untreated. Cisplatin was administered immediately prior to AS1410.

### Study Design

Mice were allocated to groups as follows:

| | | | No. |
|---|---|---|---|
| | | | of |
| Group | Cell Line | Treatment | Mice |
| 1 | A549 | Untreated control | 9 |
| 2 | A549 | AS1410 (1 mg/kg) once daily for 9 days^{a} | 9 |
| | | and Cisplatin (6 mg/kg) on day 0 and 7 | |
| 3 | A549 | AS1410 (0.3 mg/kg) once daily for 9 days^{a} | 9 |
| | | and Cisplatin (6 mg/kg) on day 0 and 7 | |
| 4 | A549 | AS1410 (1 mg/kg) once daily for 9 days^{a} | 9 |
| 5 | A549 | Cisplatin (6 mg/kg) on day 0 and 7 | 9 |

^{a}with two day interval after four days

### Clinical Signs

All animals were observed daily for signs of ill health or overt toxicity. An individual record was maintained of the clinical condition of each animal.

The xenografts were monitored for changes in appearance, texture and condition.

### Morbidity and Mortality

All animals were examined twice daily to ensure the animals were in good health.

### Bodyweight

Individual bodyweights were recorded on day of dosing and then weekly thereafter.

### Results

FIGS. 31 to 32 respectively show the results of tumour growth curves and survival curves in xenografts derived from the A549 cell line. Comparisons were made between an untreated control, AS1410 (alone), cisplatin (alone) and two combinations of AS1410 and cisplatin (in which the doses were 0.3 mg/kg and 1 mg/kg of AS1410).

FIG. 31 shows that the relative tumour volume was about 6 after approximately 43 days for the untreated control. The results for AS1410 (alone), cisplatin (alone) and the combination of AS1410 (0.3 mg/kg) and cisplatin all showed a relative tumour volume of about 5 after approximately 60 days.

However, the combination of AS1410 (1 mg/kg) and cisplatin showed a relative tumour volume of only about 2 after approximately 60 days. This shows a significant and unexpected advantage over the above.

FIG. 32 shows that there was 0% survival after about 52 days in the untreated control. This compares with a percentage survival of between about 40 and 60% after the same amount of time for AS1410 (alone), cisplatin (alone) and the combination of AS1410 (0.3 mg/kg) and cisplatin.

However, the combination of AS1410 (1 mg/kg) and cisplatin showed 100% survival even after 60 days. Again this shows a significant and unexpected advantage over the other treatment regimes.

### Example 8

### Physicochemical Assessments

### Methods

### Solubility

Thermodynamic solubility values were determined in each of 0.1M HCl, Deionised water, pH6.8 phosphate buffer, 0.9% saline and 5% dextrose.

1.25-1.5 mg of sample was weighed out into a 1 ml HPLC tube to which 0.25 ml of the dissolution media was added to allow for determination of solubility up to 5 mg/ml. This suspension was then shaken for 24 hours before measurement of the pH, filtration (through a 96 well plate with a glass fibre filter membrane), dilution and analysis of the neat and diluted solutions at different injection volumes on a generic 5 minute HPLC method.

A reference solution was prepared in DMSO for comparison and calculation of the test solubilities. XRPD patterns were measured of remaining solids from experiments where complete dissolution was not achieved.

It is believed the large additional peak noted in the HPLC chromatograms is due to a rotamer of the parent compound. This should therefore be included in the overall peak area assessment. Therefore, the largest and second largest peaks noted in the reference solution were added together as were the same peaks (i.e. the same retention times) in the test sample injections.

### X-ray Powder Diffraction (XRPD)

X-ray powder diffraction was carried out on a Bruker C2 diffractometer equipped with an XYZ stage and laser video microscope for auto-sample positioning; and a HiStar area Detector with typical collection times of 120 s. The sealed copper tube (Cu Kα radiation; 1.5407 Å) voltage and current were set at 40 kV and 40 mA. The X-ray optics on the C2 consists of a single Göbel mirror coupled with a pinhole collimator of 0.3 mm. Beam divergence i.e. effective size of X-ray spot, gives a value of approximately 4 mm. Theta-theta continuous scans were employed with a sample—detector distance of 20 cm which gives an effective 2 theta range of 3.2-29.8°. A corundum (α—Al₂O₃) standard (NIST 1976 flat plate) was run weekly to check the instrument calibration. Sample preparation consisted of a few mg of sample pressed lightly on a glass slide to obtain a flat surface.

### PKa/LogP/D

Pka determination was performed on a Sirius GlpKa instrument equipped with a D-PAS (Dip-Probe Absorption Spectroscopy) attachment. Measurements were made by potentiometric and UV titration in MeOH:H₂O mixtures at 25° C. The titration media was ionic strength adjusted with 0.15 M KCl. The values found in the MeOH:H₂O mixtures were extrapolated to 0% co-solvent via a Yasuda-Shedlovsky extrapolation. Prediction of pKa values was made using ACD v8.07 software.

The LogP values were measured by titration of solutions of the compound in Octanol/ISA (ionic strength adjusted) water mixtures on a Sirius GipKa instrument. Titrations in different ratios of Octanol to water enabled the calculation of the Log Pᵢₒₙ values and therefore the LogD profile. Prediction of LogP values were made using ACD v8.07 and Syracuse KNOWWIN v1.67 software.

Material that had precipitated from two of the pKa titrations (BSG01) and BSG17) was filtered and washed with water prior to XRPD and HPLC analysis.

### Purity Analysis

Purity analysis was performed on an Agilent HP 1100 system and the data collected and processed using Chemstation V9.0.

Total Method Duration: 12.5 minutes

Column: LUNA C18 (2) 150×4.6 mm i.d. 5 μm particle size (Phenomenex)

Column Temperature: 40° C.

Injection volume: 5 μl

Flow Rata ml/min: 0.6 ml/min

Detection: Diode array from 210-310 nm

Phase A: Methanol (Analar, HPLC Grade)+0.1% Formic Acid (HPLC grade)

Phase B: Water (HPLC grade)+1% Formic Acid (HPLC grade)

Gradient Timetable:

TABLE 6
Summary Table of HPLC Gradient method

| Time/Min | % Phase B | % Phase A |
|---|---|---|
| 0 | 90 | 10 |
| 0.5 | 90 | 10 |
| 6.5 | 10 | 90 |
| 8.0 | 10 | 90 |
| 8.5 | 90 | 10 |
| 12.5 | 90 | 10 |

### Stability

Due to the generally low solubility of these compounds in 0.9% NaCl and 5% dextrose it was decided to mix these media with 50% Iso-propanol (IPA) to maintain full dissolution of the compound during these studies. A parallel study of stability in IPA at 25° C. was also carried out to ensure that this solvent did not have a significant effect on stability.

The remaining 5 mg/ml DMSO stock solution from the pKa determination was used for the stability studies. This solution had been stored in the fridge for several days before these stability studies. A solution with a concentration of 0.1 mg/ml was prepared for each experiment (50 fold dilution of DMSO stock). Stability trials were carried out at 4° C. (fridge) and 25° C. (on top of shaker at 25° C.) and at time points of 0, 1 and 7 days. In the case of 100% IPA data was collected at 0, 1 and 14 days after storage at 25° C. The HPLC purity method discussed in section 4.5 below was used for purity analysis. The largest and second largest peaks were added together due to separation of rotamers as discussed above.

### Results of Physicochemical Tests

### Solubility

TABLE 7
Thermodynamic Solubility results

| | | | Solubility mg/ml |
|---|---|---|---|
| | | PH of unfiltered | Free Base |
| Compound ID | Test solvent | Saturated solution | equivalent |
| BSG01 | 0.1M HCl | 1.25 | >5 |
| BSG01 | Water | 8.66 | <0.001 |
| BSG01 | PH 6.8 | 7.45 | 2.63 |
| BSG01 | 0.9% NaCl | 9.80 | <0.001 |
| BSG01 | 5% Dextrose | 8.26 | 0.22 |
| BSG17 | 0.1M HCl | 1.12 | >5 |
| BSG17 | Water | 8.34 | 0.020 |
| BSG17 | pH6.8 | 7.49 | 1.95 |
| BSG17 | 0.9% NaCl | 8.81 | <0.001 |
| BSG17 | 5% Dextrose | 7.96 | 0.30 |

### PKa and LogP/D

TABLE 8
pKa predictions

| | ACD Predicted pKa | | | | | | | |
|---|---|---|---|---|---|---|---|---|
| | | | | | | | | Comment e.g. |
| | | | | | | | Version | A = Acid |
| Compound ID | PKa1 | PKa2 | PKa3 | PKa4 | PKa5 | PKa6p | Used | B = Base |
| BSG01 | 5.34 | 9.35 | 9.95 | 10.92 | 12.93 | 13.92 | V8.07 | BBBBAA |
| BSG17 | 9.35 | 9.78 | 12.89 | 13.88 | | | V8.07 | BBAA |

TABLE 9
LogP/D predictions

| | | ACD | Predicted | | |
|---|---|---|---|---|---|
| | | LogP, | LogD_{6.8} | | |
| Compound | | LogD | Version | Syracuse | Predicted LogP |
| ID | LogP | PH 6.5 | used | LogP | Version used |
| BSG01 | 4.44 | −0.31 | V8.08 | 4.14 | KOWWINv.1.67 |
| BSG17 | 4.48 | −0.62 | V8.08 | 4.10 | KOWWINv.1.67 |

TABLE 10
pKa measurements

| | Measured pKa | | | | | |
|---|---|---|---|---|---|---|
| Compound | | | | | PH Metric | Co- |
| ID | pKa1 | pKa2 | pKa3 | pKa4 | or UV | solvent? |
| BSG01 | 4.15 UV | 8.14 | 9.08 | 9.31 | UV + Pot | No + Yes |
| BSG17 | 8.17 | 9.09 | 9.61 | | UV + Pot | No + Yes |

### Comments on Table 10

BSG01-4.14 from aqueous UV measurement (Potentiometry gave 4.15 although incomplete curves were collected). Other values from potentiometric in Aqueous/MeOH mixtures followed by Yasuda-Shedlovsky extrapolation to 0% MeOH. All pKas basic from slope of extrapolation. P BSG17—Values from potentiometric titrations in Aqueous/MeOH mixtures followed by Yasuda-Shedlovsky extrapolation to 0% MeOH. All pKas basic from slop of extrapolation. PKas 2 and 3 for titration in 30% MeOH removed from YS extrapolation due to precipitation.

TABLE 11
LogP/D measurements

| | | | | LogPᵢₒₙ | |
|---|---|---|---|---|---|
| Compound | | Measured | LogP, | LogD | LogD |
| ID | LogP | LogPᵢₒₙ | pH 6 | pH 6.5 | pH 7.4 |
| BSG01 | 5.02 | 2.62 | −2.56 | −1.47 | 0.74 |
| BSG17 | 5.54 | 3.75 | −0.15 | 0.38 | 1.58 |

### Purity Analysis

TABLE 12
Initial HPLC purity and purity of precipitate from pKa assay

| | | | | | Purity | Purity | |
|---|---|---|---|---|---|---|---|
| | Solvent/ | | Vol | | larger | smaller | Combined |
| Compound | test | Wt for | made up | Conc | rotamer | rotamer | rotamer |
| ID & test | media | mg | (ml) | mg/ml | peak | peak | purity |
| BSG 01 | Dissolved | 0.224 | 1.120 | 0.200 | 89.8 | 5.0 | 94.8 |
| Purity | in Water | | | | | | |
| | & 3 μl | | | | | | |
| | HCl | | | | | | |
| BSG 17 | Dissolved | 0.169 | 0.845 | 0.200 | 61.3 | 28.0 | 89.3 |
| Purity | in Water | | | | | | |
| | & 3 μl | | | | | | |
| | HCl | | | | | | |
| BSG 01 | Dissolved | 0.179 | 0.895 | 0.200 | 87.6 | 4.9 | 92.5 |
| Precipitate | in Water | | | | | | |
| | & 3 μl | | | | | | |
| | HCl | | | | | | |
| BSG 17 | Dissolved | 0.196 | 0.980 | 0.200 | 46.9 | 23.0 | 69.9 |
| Precipitate | in Water | | | | | | |
| | & 3 μl | | | | | | |
| | HCl | | | | | | |

### Stability

TABLE 13
BSG01 HPLC stability data

| | | | Purity | Purity | |
|---|---|---|---|---|---|
| | | | larger | smaller | Combined |
| Solvent/ | Time | Temp | rotamer | rotamer | rotamer |
| Test media | Days | ° C. | peak | peak | purity |
| 100% IPA | 0 | | 86.4 | 5.5 | 91.9 |
| 100% IPA | 1 | 25 | 87.2 | 6.0 | 93.2 |
| 100% IPA | 14 | 25 | 82.8 | 11.2 | 94.0 |
| 5% Dextrose | 1 | 4 | 84.5 | 10.00 | 94.5 |
| 5% Dextrose | 7 | 4 | 48.4 | 30.9 | 79.3 |
| 5% Dextrose | 0 | | 88.7 | 6.1 | 94.8 |
| 5% Dextrose | 1 | 25 | 79.5 | 15.0 | 94.5 |
| 5% Dextrose | 7 | 25 | 352 | 32.8 | 68.0 |
| 0.9% NaCl | 1 | 4 | 86.8 | 8.9 | 95.7 |
| 0.9% NaCl | 7 | 4 | 47.5 | 19.4 | 66.8 |
| 0.9% NaCl | 0 | | 89.8 | 5.7 | 95.4 |
| 0.9% NaCl | 1 | 25 | 82.3 | 11.2 | 93.5 |
| 0.9% NaCl | 7 | 25 | 35.1 | 12.5 | 47.6 |

All 5% Dextrose and 0.9% NaCl experiments mixed with 50% IPA to ensure complete dissolution

TABLE 14
BSG17 HPLC stability data

| | | | Purity | Purity | |
|---|---|---|---|---|---|
| | | | larger | smaller | Combined |
| Solvent/ | Time | Temp | rotamer | rotamer | rotamer |
| Test media | Days | ° C. | peak | peak | purity |
| 100% IPA | 0 | | 58.8 | 25.8 | 84.6 |
| 100% IPA | 1 | 25 | 56.6 | 25.0 | 81.5 |
| 100% IPA | 14 | 25 | 52.7 | 24.5 | 77.2 |
| 5% Dextrose | 1 | 4 | 56.9 | 26.9 | 83.8 |
| 5% Dextrose | 7 | 4 | 32.5 | 20.7 | 53.2 |
| 5% Dextrose | 0 | | 60.7 | 27.8 | 88.5 |
| 5% Dextrose | 1 | 25 | 52.4 | 25.6 | 78.0 |
| 5% Dextrose | 7 | 25 | 22.3 | 17.1 | 39.4 |
| 0.9% NaCl | 1 | 4 | 58.4 | 26.8 | 85.1 |
| 0.9% NaCl | 7 | 4 | 39.2 | 21.8 | 61.0 |
| 0.9% NaCl | 0 | | 61.4 | 28.0 | 89.4 |
| 0.9% NaCl | 1 | 25 | 55.9 | 26.5 | 82.4 |
| 0.9% NaCl | 7 | 25 | 33.8 | 20.8 | 54.5 |

All 5% Dextrose and 0.9% NaCl experiments mixed with 50% IPA to ensure complete dissolution

### Discussion

Solubility measurements (see Table 7) in 0.1M HCl gave values of greater than 5 mg/ml for both compounds due to protonation of the basic functionalities. Solubility in water was variable with BSG01 giving <0.001 mg/ml and BSG17 giving 0.02 mg/ml. It was also noted that 5% dextrose gave better solubility than 0.9% NaCl for both compounds. The errors in these measurements are higher than normal due to the effect of rotamers noted in the HPLC analysis and discussed above.

XRPD analysis of all samples (FIGS. 12 and 13) as received (i.e. prior to solubilisation and/or other testing) showed them all to be essentially amorphous.

Undissolved material from the solubility analyses was extracted from the filter plate for XRPD analysis (FIGS. 14 to 17). For both BSG01 and BSG17 there was enough extractable material to make analysis possible. It was clear that equilibration over 24 hrs in aqueous media converts the amorphous form into a crystalline state.

Crystalline forms of BSG01 and BSG17 were also formed at the end of the potentiometric pKa titration in Methanol/Water/0.15M KCl at approx. pH 11.2. XRPD analysis of these materials (FIGS. 16 and 17) showed a high degree of crystallinity. Subsequent HPLC analysis of these crystalline materials (Table 12) gave additional peaks due to rotamers as noted in the purity analysis.

The predictions and measurements of pKa and LogP/D are shown in tables 8 and 9. The measured pKa and Log P/D are shown in tables 10 and 11 and comments regarding the measurements are provided immediately following the table. The rotamers did not seem to effect the measurements of pKa and LogP with the data generated being of high quality.

FIG. 18 shows the chromatograms from the purity analysis of these materials (purity data in table 12). Final purity values are a summation of the two largest peaks which are assumed to be due to the rotamers.

Tables 13 and 14 and FIG. 19 show the stability data and plots in 50:50 IPA:5% Dextrose, 50:50 IPA 0.9% NaCl and 100% IPA. These experiments were done in 50:50 IPA/test media mixtures to ensure complete dissolution of the material. Therefore stability data in 100% IPA was also generated for comparison to ensure IPA did not have a major impact compared to the test media. Stability tests in IPA for 14 days at 25° C. showed very little change for all the samples. Temperature did not have any significant effect on stability and 5% dextrose and 0.9% NaCl gave very similar results. All samples had degraded significantly after 7 days at 4° C. and 25° C.

### Example 9

### Pharmaceutical Formulations and Administration

The compounds of the invention may be formulated into a pharmaceutical formulation comprising a compound according to the first aspect of the invention in admixture with a pharmaceutically or veterinarily acceptable adjuvant, diluent or carrier.

The formulation is a unit dosage containing a daily dose or unit, daily sub-dose or an appropriate fraction thereof, of the active ingredient.

The compounds of the invention will normally be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the compounds of the invention can be administered alone but will generally be administered in admixture with a suitable pharmaceutical excipient, adjuvant, diluent or carrier selected with regard to the intended route of administration and standard pharmaceutical practice.

For example, the compounds of the invention can be administered orally, buccally or sublingually in the form of tablets, capsules, ovules, elixirs, solutions or suspensions, which may contain flavouring or colouring agents, for immediate-, delayed- or controlled-release applications. The compounds of invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatine and acacia. Additionally, lubricating agents such as magnesium stearate, stearic acid, glyceryl behenate and talc may be included.

Solid compositions of a similar type may also be employed as fillers in gelatine capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The compounds of the invention can also be administered parenterally, for example, intravenously, intra-arterially, intraperitoneally, intrathecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of the compounds of the invention will usually be from 1 mg/kg to 30 mg/kg. Thus, for example, the tablets or capsules of the compounds of the invention may contain a dose of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The compounds of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A, or 1,1,1,2,3,3,3-heptafluoropropane (HFA 227EA_{c}), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatine) for use in an inhaler or insufflator may be formulated to contain a powder mix of a compound of the invention and a suitable powder base such as lactose or starch.

Aerosol or dry powder formulations are preferably arranged so that each metered dose or “puff” delivers an appropriate dose of a compound of the invention for delivery to the patient. It will be appreciated that the overall daily dose with an aerosol will vary from patient to patient, and may be administered in a single dose or, more usually, in divided doses throughout the day.

Alternatively, the compounds of the invention can be administered in the form of a suppository or pessary, or they may be applied topically in the form of a lotion, solution, cream, ointment or dusting powder. The compounds of the invention may also be transdermally administered, for example, by the use of a skin patch. They may also be administered by the ocular route, particularly for treating diseases of the eye.

For ophthalmic use, the compounds of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatine and glycerin, or sucrose and acacia; and mouth-washes comprising the active ingredient in a suitable liquid carrier.

Generally, in humans, oral or topical administration of the compounds of the invention is the preferred route, being the most convenient. In circumstances where the recipient suffers from a swallowing disorder or from impairment of drug absorption after oral administration, the drug may be administered parenterally, e.g. sublingually or buccally.

For veterinary use, a compound of the invention is administered as a suitably acceptable formulation in accordance with normal veterinary practice and the veterinary surgeon will determine the dosing regimen and route of administration which will be most appropriate for a particular animal.

### REFERENCES

Each of the following disclosures is incorporated herein by reference in its entirety into the present disclosure.
1. Autexier, 1999, “Telomerase as a Possible Target for Anticancer Therapy,” Chemistry & Biology, November 1999, Vol 6, ppR299-R303
2. Bogert et al, 1930, Collect Czech Chem Comm., Vol 2, pp 383-395
3. Bostock-Smith et al., 1988, “Anti-tumour Polycyclic Acridines, Part 6,” Biochemistry, Vol 38, No 21, pp 6723-6731
4. Gimenez-Arnau et al., 1998, “Anti-tumour Polycyclic Acridines, Part 2,” Anti-Cancer Drug Design, Vol 13, pp 125-143
5. Gimenez-Arnau et al., 1998, “Anti-tumour Polycyclic Acridines, Part 4,” Anti-Cancer Drug Design, Vol 13, pp 431-451
6. Goldberg and Kelly, 1946, J. Chem. Soc., p 102
7. Goldstein and de Simo, 1927, Helv. Chim. Acta., Vol 10, p 604
8. Hagan et al., 1997, “Anti-tumour Polycyclic Acridines, Part 1,” J Chem Soc., Perkin TransI, p 2739
9. Hagan et al., 1998, “Anti-tumour Polycyclic Acridines, Part 3,” J Chem Soc., Perkin TransI, p 915
10. Harrison et al., 1999, “Human Telomerase Inhibition by Substituted Acridine Derivatives,” Bioorganic & Medicinal Chemistry Letters, Vol 9, pp 2463-2468
11. Julino et al., 1998, “Anti-tumour Polycyclic Acridines, Part 5,” J Chem Soc., Perkin TransI, p 1677
12. Korolev et al., 1976, J Gen Chem USSR (Engl Trans.), Vol 46, pp 2250-2252
13. Korolev et al, 1977, J Gen Chem USSR (Engl Trans.), Vol 47, pp 2118-2122
14. Matsumura, 1929, Journal, Vol V, NoN, ppP-P
15. Neidle et al., 1999, “Telomerase as an Anti-Cancer Target: Current Status and Future Prospects,” Anti-Cancer Drug Design, Vol 14, pp 341-347
16. Perry et al., 1998a, “1,4- and 2,6-Disubstituted Amidoanthracene-9,10-dione Derivatives as Inhibitors of Human Telomerase,” J Med Chem., Vol 41, pp 3253-3260
17. Perry et al., 1998b, “Human Telomerase Inhibition by Regioisomeric Disubstitued Amidoanthracene-9,10-diones,” J Med Chem., Vol 41, No 24, pp 4873-4884
18. Perry et al., 1998c, “Telomeres and Telomerase: Targets for Cancer Chemotherapy?,” Exp Opin Ther Patents, Vol 8, No 12, pp 1567-1586
19. Perry et al., 1999a, “Design, Synthesis and Evaluation of Human Telomerase Inhibitors Based Upon a Tetracyclic Structural Motif,” Anti-Cancer Drug Design, Vol 14, pp 373-382
20. Perry et al., 1999b, “2,7-Disubstituted Amidofluorenone Derivatives as Inhibitors of Human Telomerase,” J Med Chem., Vol 42, No 14, pp 2679-2684
21. Read et al, Apr. 24, 2001, “Structure-based design of selective and potent G quadruplex-mediated telomerase inhibitors,” PNAS, Vol 98, No 9, pp 4844-4849
22. Sharma et al., 1997, “Preclinical and Clinical Strategies for Development of Telomerase and Telomere Inhibitors,” Annals of Oncology, Vol 8, pp 1063-1074
23. Sun et al., 1997, “Inhibition of Human Telomerase by a G-Quadruplex-Interactive Compound,” J Med Chem., Vol 40, pp 2113-2116
24. Urquidi et al., 1998, “Telomerase in Cancer: Clinical Applications,” Ann Med., Vol 30, pp 419-430.
25. Burger et al., 2005, “The G-quadruplex-interactive molecule BRACO-19 inhibits tumor growth, consistent with telomere targeting and interference with telomerase function”, Cancer Res, Vol 65, pp 1489-1496.
26. Incles et al., 2004, “A G-quadruplex telomere targeting agent produces p 16-associated senescence and chromosomal fusions in human prostate cancer cells”, Mol Cancer Therapeutics, Vol 3, pp 1201-1206.
27. Schultes et al., 2004, “Synthesis, biophysical and biological evaluation of 3,6-bis-aminoacridines with extended 9-anilino substituents as potent G-quadruplex-binding telomerase inhibitors”, Bioorg Med Chem Letters, Vol 14, pp 4347-4351.
28. Harrison et al., 2003, “Trisubstituted acridine derivatives as potent and selective telomerase inhibitors”, J Med Chem., Vol 46, pp 4463-4476
29. Neidle and Parkinson, 2002, “Telomere maintenance as a target for anticancer drug discovery”, Nat Rev Drug Discovery, Vol 1, pp 383-393
30. Parkinson, Lee and Neidle, 2002, “Crystal structure of parallel quadruplexes from human telomeric DNA”, Nature, Vol 417, pp 876-880
31. Shay and Wright, 2002, “Telomerase: a target for cancer therapeutics”, Cancer Cell, Vol 2, pp 257-265
32. Riou, 2004, “G-quadruplex interacting agents targeting the telomeric G-overhang are more than simple telomerase inhibitors”, Curr Med Chem Anti-Canc Agents, Vol 4, pp 439-443
33. Mergny et al., 2002, “Natural and pharmacological regulation of telomerase”, Nucleic Acids Res, Vol 30, pp 839-865
34. Leonetti et al., 2004, “Biological activity of the G-quadruplex ligand RHPS4 (3,11-difluoro-6,8,13-trimethyl-8H-quino[4,3,2-kl]acridinium methosulfate) is associated with telomere capping alteration”, Mol Pharmacology, Vol 66, pp 138-1146
35. Guyen et al., 2004, “Synthesis and evaluation of analogues of 10H-indolo[3,2-b]quinoline as G-quadruplex stabilising ligands and potential inhibitors of the enzyme telomerase”, Org Biomol Chem, Vol 2, pp 981-988

1-33. (canceled)

34. A compound of formula I: 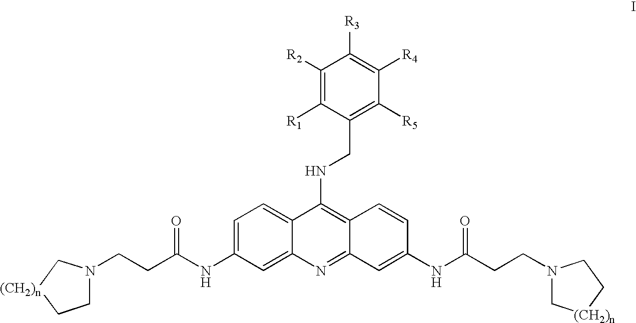
wherein each of R₁, R₂, R₃, R₄ and R₅ is either fluorine or hydrogen;
n represents 1 or 2; and
which compound is optionally substituted at one or more positions by substituents independently selected from:
halo; hydroxy; ether; imino; oxo; formyl; acyl; acylhalide; carboxy; ester; acyloxy; amido; acylamido; thioamido; tetrazolyl; amino; nitro; nitroso; azido; cyano; isocyano; cyanato; isocyanato; thiocyano; isothiocyano; sulfhydryl; thioether; sulphonic acid; sulphonate; sulphone; sulphonyloxy; sulphinyloxy; sulphamino; sulphonamino; sulphinamino; sulphamyl; sulphonamido; C₁₋₇alkyl; C₃₋₂₀heterocyclyl; and C₅₋₂₀aryl,
or a pharmaceutically acceptable derivative thereof.

35. The compound of claim 34, wherein the compound is represented by the following structural formula: 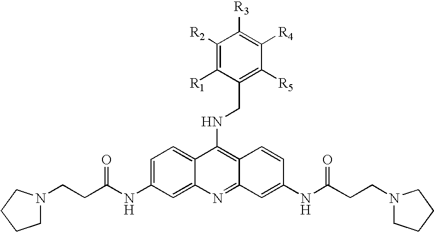
or a pharmaceutically acceptable derivative thereof.

36. The compound of claim 35, wherein the compound is represented by the following structural formula: 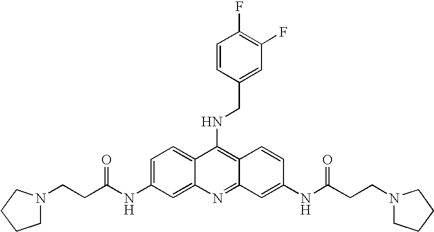
or a pharmaceutically acceptable derivative thereof.

37. The compound of claim 34, wherein the compound is in a prodrug form.

38. A pharmaceutical composition comprising a compound as claimed in claim 34 and a pharmaceutically acceptable excipient, adjuvant, diluent or carrier.

39. A method of inhibiting telomerase in a subject in need thereof, comprising administering to the subject an effective amount of the compound of claim 34.

40. A method of regulating cell proliferation in a subject in need thereof, comprising administering to the subject an effective amount of the compound of claim 34.

41. A method of treating and/or preventing and/or diagnosing a disease characterized by increased cell proliferation in a subject, comprising administering an effective amount of the compound of claim 34.

42. The method of claim 41, wherein the disease characterized by increased cell-proliferation is a benign, pre-malignant, or malignant cellular proliferation.

43. The method of claim 41, wherein the disease is selected from the group of neoplasm and tumors, cancer, leukemia, psoriasis, bone disease, fibroproliferative disorders, and atherosclerosis.

44. The method of claim 43, wherein the disease is cancer.

45. The method of claim 43, wherein the disease is a tumor selected from the group consisting of histocytoma, glioma, astrocytoma and osteoma; or the disease is a cancer selected form the group consisting of ovarian carcinoma, breast carcinoma, bowel cancer, colon cancer, renal cancer, lung cancer, small cell lung cancer, testicular cancer, prostate cancer, sarcoma, osteosarcoma, Kaposi's sarcoma, melanoma, leukemias, pancreatic cancer and skin cancer.

46. The method of claim 41 wherein the actions of the compound are characterized by increased cell proliferation associated with an activity selected from the group consisting of the regulation of cell proliferation; the inhibition of angiogenesis; the inhibition of metastasis; the inhibition of invasion of tumor cells into neighboring normal structures; or the promotion of apoptosis.

47. The method of claim 41, wherein the compound is administered as a plurality of doses.

48. The method of claim 41, wherein the effective amount is between 1 and 500 mg/m².

49. A method of inhibiting telomerase comprising contacting a cell with an effective amount of a compound as defined in claim 34.

50. A method of regulating cell proliferation comprising contacting a cell with an effective amount of a compound as defined in claim 34.

51. The method of claim 49, wherein the amount of compound or composition is 0.1 to 10 micromolar.

52. The method of claim 50, wherein the amount of compound or composition is 0.1 to 10 micromolar.

53. A method of preparing a compound in claim 34 comprising the step of reacting a compound of formula II, 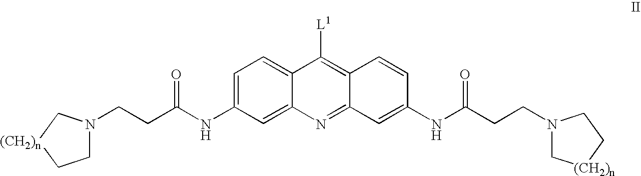
wherein L¹ represents a suitable leaving group and n is as defined in claim 34, with a compound of formula III, 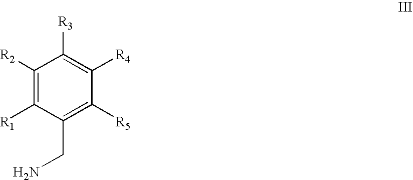
wherein R₁ to R₅ are as defined in claim 34.

54. A method of preparing the compound of claim 34 comprising the steps shown in FIG. 1.

55. A kit comprising:
(a) the compound as defined in claim 34; and
(b) instructions for use.

56. The kit as claimed in claim 55 wherein the compound is provided in a suitable container and/or with suitable packaging.

57. The kit as claimed in claim 55 wherein the instructions are written instructions on how to administer the active compound.

58. The kit as claimed in claims 55 wherein the instructions for use include a list of indications for which the active ingredient is a suitable treatment.

59. The kit as claimed in claims 55 comprising a means of administering the compound and/or composition.

60. A combination product comprising:
(A) the compound of Formula I as defined in claim 34 or a compound of formula Ia, as defined below, or a pharmaceutically acceptable derivative thereof; and
(B) a platin, or a pharmaceutically acceptable derivative thereof, wherein each of components (A) and (B) is formulated in admixture with a pharmaceutically acceptable adjuvant, diluent or carrier,
wherein the compound of formula Ia has the following structure: 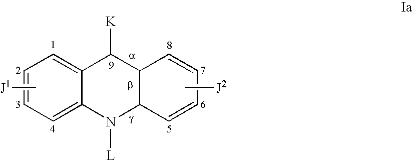
wherein either:
(a) K is O, L is —H, α is a single bond, β is a double bond, γ is a single bond; or:
(b) K is a 9-substituent, L is absent, α is a double bond, β is a single bond, γ is a double bond; and
J¹ and J² independently represent —N(H)—C(O)—(CH₂)₂—NR^{1a}R^{2a}:
n represents an integer from 1 to 5;
K represents —N(R^{N})Q;
R^{1a}, R^{2a} and R^{N} independently represent H, C₁₋₇alkyl, C₃₋₂₀heterocyclyl or C₅₋₂₀aryl, which latter three groups may be optionally substituted by one or more substituents selected from unsubstituted C₁₋₇alkyl, C₃₋₂₀heterocyclyl, C₅₋₂₀aryl and the list of optional substituents hereinbefore defined in respect of compounds of formula I; and
Q represents C₁₋₇alkyl, C₃₋₂₀heterocyclyl or C₅₋₂₀aryl, all of which may be optionally substituted as defined above,
and pharmaceutically acceptable derivatives thereof.

61. The combination product of claim 60 comprising a pharmaceutical formulation including the compound of formula I, the compound of formula Ia, or a pharmaceutically acceptable derivative thereof; or a platin, or a pharmaceutically acceptable derivative thereof;
and a pharmaceutically acceptable adjuvant, diluent or carrier.

62. The combination product of claim 60 which comprises a kit of parts comprising components:
(A) a pharmaceutical formulation including the compound of formula I or the compound of formula Ia, or a pharmaceutically acceptable derivative thereof, in admixture with a pharmaceutically acceptable adjuvant, diluent or carrier; and
(B) a pharmaceutical formulation including a platin, or a pharmaceutically acceptable derivative thereof, in admixture with a pharmaceutically acceptable adjuvant, diluent or carrier; and
wherein each of components (A) and (B) is formulated in admixture with a pharmaceutically acceptable adjuvant, diluent or carrier.
